(19) United States
(12) Patent Application Publication
(10) Pub. No.: US 2002/0054924 A1
(43) Pub. Date: May 9, 2002

(54) Novel compositions derived from cranberry and grapefruit and therapeutic uses therefor

(76) Inventors: Margaret M. Leahy, Pocasset, MA (US); Martin Starr, Plymouth, MA (US); Elzbieta Kurowska, London (CA); Najla Guthrie, London (CA)

Correspondence Address: LAHIVE & COCKFIELD, 28 STATE STREET, BOSTON, MA, 02109, US

(21) Appl. No.: 09/835,121

(22) Filed: Apr. 13, 2001

Related U.S. Application Data
(60) Provisional application No. 60/196,886, filed on Apr. 13, 2000.

(51) Int. Cl. A61K 35/78

(52) U.S. Cl. 424/732; 424/736

(57) ABSTRACT

Novel compositions derived from grapefruit and cranberry are disclosed, as well as therapeutic uses for the compositions in treating or preventing cancer and hypercholesterolemia in a subject. The compositions are, in particular embodiments, derived from grapefruit essence oil, grapefruit peel oil, grapefruit peel, and decharacterized cranberry fruit.

### RELATED INFORMATION

This application claims priority to U.S. Provisional Application No. 60/196,886, entitled “NOVEL COMPOSITIONS DERIVED FROM CRANBERRY AND GRAPEFRUIT AND THERAPEUTIC USES THEREFOR,” filed on Apr. 13, 2000, incorporated herein in its entirety by this reference. The contents of all patents, patent applications, and references cited throughout this specification are hereby incorporated by reference in their entireties.

### BACKGROUND OF THE INVENTION

For millennia, humankind has relied on plant derivatives for the prevention and treatment of a wide variety of aliments. For example, in China, various teas have been used as a crude medicine for over 4,000 years. And more recently, there has been considerable interest in taking advantage of various plant extracts as a source of health promoting substances such as, natural oxidants, phenolic compounds, flavonoids, tocochromanols, and beneficial fatty acids. In part, this trend is due to a growing body of evidence demonstrating that some of these compounds have beneficial properties that may be advantageous in preventing or delaying the onset of disease.

Indeed, several epidemiological studies considering the affect of diet on disease such as, e.g., cancer and hypercholesterolemia, have provided leads in the search for naturally-occurring anti-cancer or anti-cholesterol agents. For example, some studies suggest that plant-based diets, rich in whole grains, legumes, fruits and vegetables, may reduce the risk of various types of cancer, including breast cancer (Steinmetz & Potter, 1991).

Similarly, other studies indicate that populations consuming large amounts of cereal grains have lower cholesterol levels and a lower incidence of cardiovascular disease. These studies have attributed these beneficial properties of cereal diets on the presence of naturally occurring tocochromanols, and these compounds have been found in a wide variety of plant sources (Quereshi et al., Am. J. Clin. Nutr., 53:1021 S-6S (1991)).

Moreover, additional studies suggest that fruit products are a source of a number of health promoting phytochemicals (Johns et al., Recent Advances in Phytochemistry, pp.31-52, Plenum Press (1997)).

Given that cancer and cholesterol-related diseases (e.g., arteriosclerosis) are two of the major causes of death in the United States, additional research on and identification of fruit-derived therapeutic compounds which, for example, are useful in treating or preventing such diseases, would be of great benefit.

### SUMMARY OF THE INVENTION

The present invention provides novel compounds and therapeutic compositions (i.e., formulations) derived from fruits, particularly cranberry and grapefruit, as well as novel uses for the compounds and compositions. In particular embodiments, the compounds are formulated as a pharmaceutical, a foodstuff (e.g., added to a foodstuff to enhance its nutritional and/or medical value), or a dietary supplement. In all cases, the compounds and compositions contain, or are enriched for, health promoting components (e.g., phenolics, flavonoids, tocochromanols) that are useful in treating or preventing a variety of health-related disorders and diseases.

Accordingly, in another embodiment, the present invention provides a method for treating or preventing a disease in a subject, particularly a malignancy (e.g., a cancer), by administering to the subject (e.g., orally or by injection) a therapeutically-effective amount of a compound or composition of the invention. The malignancy can be, for example, metastatic, breast cancer, or metastatic breast cancer. In another embodiment, the present invention provides a method for treating or preventing hypercholesterolemia in a subject, by administering to the subject a therapeutically-effective amount of a compound or composition of the invention. The hypercholesterolemia can be measured or detected by, for example, altered apoB levels.

Novel compositions of the invention are derived (e.g., isolated) from, or contain components of citrus or cranberry fruits. A preferred citrus fruit for use in the invention is grapefruit, including pink grapefruit, red peel grapefruit and combinations thereof. Particular compositions identified by way of the present invention as having significant therapeutic value include and/or are derived from essence oil and peel oil isolated from citrus fruit (e.g., grapefruit), peel (e.g., in processed form) isolated from a citrus fruit (e.g., grapefruit), decharacterized cranberry fruit (e.g., presscake), and combinations thereof. Such compositions, and compounds derived therefrom, can then be formulated in a variety of manners, such as a dietary supplement, a pharmaceutical, or as an additive to a foodstuff. They may also include additional desirable compounds further contain fats, carbohydrates, proteins, vitamins, minerals and combinations thereof.

In a related embodiment, the present invention further provides therapeutic compositions containing novel combinations and/or ratios of health-promoting compounds derived (e.g., isolated) from grapefruit and cranberry. Such compounds can be isolated from, for example, grapefruit essence oil, grapefruit peel and/or peel oil, grapefruit juice, decharacterized cranberry fruit and cranberry juice. By way of illustration, such compounds can include phenolic acids, flavanoids, fibers, omega-3-fatty acids, tocochromanols, triterpenoids, ellagic acids or combinations thereof.

In a particular embodiment, the invention provides a composition containing an anthocyanin, a phenolic acid, a proanthocyanidin, or a combination thereof. In a preferred embodiment, the anthocyanin content is 30% or greater of that present in the native fruit, the total phenolic acid content is 8% or greater of that present in the native fruit, and the proanthocyanidin content is 60% or greater of that present in the native fruit. Particular anthocyanins may include, for example, cyanidin-3-arabinoside, cyanidin-3-galactoside, cyanidin-3-glucoside, peonidin-3-arabinoside, peonidin-3- galactoside, peonidin-3-glucoside, malvidin-3-arabinoside, malvidin-3-glucoside, or combinations thereof. Particular phenolic acids may include, for example, para-coumaric acid, caffeic acid, chlorogenic acid, ferulic acid, protocatechuic acid, cinnamic acid, benzoic acid, gallic acid, para-hydroxybenzoic acid, and combinations thereof. Particular proanthocyanidins may include, for example, flavan-3-ol polymers, procyanidin B1, procyanidin B2, procyanidin B3, epicatechin oligomers, and combinations thereof. Particular flavonoids may include, for example, proanthocyanidin, flavan-3-ol, anthocyanin, flavanol, and combinations thereof. Particular flavan-3-ols may include, for example, catechin, catechin gallate, epicatechin, epicatechin gallate, epigallocatechin gallate, gallocatechin gallate, and combinations thereof. Particular flavanols may include, for example, quercetin, q-3-arabinoside (avicularin), q-3-galactoside (hyperin), q-3-glucoside (isoquercitrin), q-3 -rhamnoside (quercitrin), myricetin, m-3-arabinoside, m-3-rhamnoside (myricitrin), m-3-digalactoside, kaempferol, isorhamnetin, and combinations thereof. Particular, triterpenoids include, for example, ursolic acid.

Other features and advantages of the invention will be apparent from the following detailed description and claims.

### BRIEF DESCRIPTION OF THE DRAWINGS

FIG. 1 is a graph showing the anti-hypercholesterolemia effects of increasing amounts of grapefruit essence oil (OS3) or grapefruit red peel oil (OS4) resulting in reduced apoB secretion in liver cells (HepG2).

FIG. 2 is a graph showing that the overall normal metabolism and growth of cancer-inoculated nude mice fed four different grapefruit or cranberry based diets (concentrated pink grapefruit juice, processed grapefruit peel, concentrated cranberry juice, and decharacterized cranberry) was essentially unaltered compared to control mice.

FIG. 3 is a graph showing the reduced tumor incidence (lung metastases) in test animals administered four different grapefruit or cranberry based diets (concentrated pink grapefruit juice, processed grapefruit peel, concentrated cranberry juice, and decharacterized cranberry) after inoculation with a cancer.

FIG. 4 is a graph showing reduction in tumor size in test animals administered certain grapefruit or cranberry based diets (concentrated pink grapefruit juice, processed grapefruit peel, concentrated cranberry juice, and decharacterized cranberry) after inoculation with a cancer.

FIG. 5 is a graph showing the reduced tumor incidence (lymph node metastases) in test animals administered four different grapefruit or cranberry based diets (concentrated pink grapefruit juice, processed grapefruit peel, concentrated cranberry juice, and decharacterized cranberry) after inoculation with a cancer.

FIG. 6 is a graph showing the reduced tumor incidence (rates of both lung and lymph node metastases are compared) in test animals administered certain grapefruit or cranberry based diets (listed in order of increasing effectiveness: processed grapefruit peel, concentrated pink grapefruit juice, concentrated cranberry juice, and decharacterized cranberry).

### DETAILED DESCRIPTION OF THE INVENTION

In order to provide a clear and consistent understanding of the specification and claims, including the scope to be given such terms, the following definitions are provided.

Definitions

The term “cancer” refers to any neoplasm (e.g., benign or malignant), such as a carcinoma (i.e., usually derived from epithelial cells, e.g., breast cancer) or sarcoma (usually derived from connective tissue cells, e.g., a bone or muscle cancer) or a cancer of the blood, such as a erythroleukemia (a red blood cell cancer) or leukemia (a white blood cell cancer). “Malignant” cancers (i.e., malignancies) are generally metastatic, i.e., have acquired the ability to transfer from one organ or tissue to another not directly connected, e.g., through the blood stream or lymphatics.

The term “anti-cancer activity” or “anti-cancer properties” refers to the inhibition (in part or in whole) or prevention of a cancer as defined herein. Anti-cancer activity includes, e.g., the ability to reduce, prevent, or repair genetic damage, modulate undesired cell proliferation, modulate misregulated cell death, or modulate mechanisms of metastasis (e.g., ability to migrate).

The term “hypercholesterolemia” refers to abnormally high serum levels of cholesterol, typically due to defective cholesterol metabolism in a subject.

The term “anti-hypercholesterolemic activity” refers to the ability to regulate cholesterol metabolism or reduce serum cholesterol levels in a subject.

The term “aroma” refers to the water-soluble components (e.g., fraction) remaining after evaporation of a fruit juice.

The term “essence oil” refers to the oil-soluble components (e.g., fraction) remaining after evaporation of a fruit juice.

The term “peel oil” refers to oil isolated from the peel of a citrus fruit.

The term “peel” refers to the peel of a citrus fruit which, for purposes of the present invention, may be e.g., dried, shredded, or pelletized.

The term “citrus fruit” refers to a fruit from the genus Citrus that includes, e.g., orange, lemon, lime, tangerine and, in particular, grapefruit (e.g., pink grapefruit, red peel grapefruit).

The term “decharacterized fruit” refers to fruit from which the juice has been extracted. The decharacterized fruit can be in the form of, for example, a mash or presscake. The term “Tomah presscake” refers to a particularly preferred presscake described in U.S. Pat. Nos. 5,320,861 and 5,320,861 which contains higher levels of desirable phytochemicals than are present in presscake made via conventional methods. In particular, decharacterized cranberry fruit in the form of “Tomah presscake” contains higher levels of anthocyanins, phenolic acids and proanthocyanidins than that found in presscake produced through conventional methods. For example, the anthocyanin content is typically 30% or greater of that present in native cranberry fruit, the phenolic acid content is typically 8% or greater of that present in native cranberry fruit and the proanthocyanidin content is typically 60% or greater of that present in native cranberry fruit.

The term “isolated” refers to the removal or change of a composition or compound from its natural context.

The term “phenolic compound” refers to a compound that is an aromatic acid having one or more hydroxyl groups on the benzene ring and naturally present at some measurable level in a fruit. In a preferred embodiment, the phenolic compounds of the invention include, e.g., para-coumaric acid, caffeic acid, chlorogenic acid, ferulic acid, protocatechuic acid, cinnamic acid, benzoic acid, gallic acid, para-hydroxybenzoic acid, or a combination thereof.

The term “flavonoid” refers to any member of the group of aromatic, oxygen- containing, heterocyclic pigments found in the derivatives of the invention and includes for example members of the chemical subgroups 1) catechins, 2) leucoanthocyanidins and flavanones, 3) flavanins, flavones, and anthocyanins, and 4) flavonols. In preferred embodiments, a flavonoid includes, e.g., a proanthocyanidin, flavan-3-ol, anthocyanin, or flavanol.

The term “fiber” includes the principal chemical class of dietary fiber, which includes cellulose, hemicelluloses, gums, lignins, and preferably, pectins.

The term “fatty acid” refers to a fatty acid that is naturally present at some measurable level in the fruit derivatives of the invention and includes, for example, a- linolenic acid (omega-3), oleic acid (omega-9), linoleic acid (omega-6), or a combination thereof.

The term “tocochromanol” refers to any tocopherol (T) or tocotrienol (T3) compound, for example, α-tocopherol, γ-tocopherol, δ-tocopherol, α-tocotrienol, γ-tocotrienol, δ-tocotrienol, or a combination thereof, that is present in measurable levels in the fruit derivatives of the invention.

The term “triterpenoids” refers to components such as, e.g., ursolic acid, that are naturally present at some measurable level in the fruit derivatives of the invention.

The term “ellagic acid” refers to a plant phenol that is naturally present at some measurable level in the fruit derivatives of the invention.

The term “foodstuff” refers to any edible substance that can be used as or in food for an animal or human. Foodstuffs include substances that may be used in the preparation of foods such as cooking oils or food additives. Foodstuffs also include dietary supplements designed to, e.g., supplement the diet of an animal or human. In addition, foodstuffs also include animals or animal products used for human consumption, such as, for example eggs or milk. Such animal themselves can be fed or treated with a composition of the invention and retain the advantageous properties of the composition (e.g., decharacterized cranberry or components thereof) or impart those advantageous properties to products such as eggs or milk.

The term “pharmaceutical composition” or “therapeutic composition” refers to a composition formulated for therapeutic use.

The terms “health promoting”, “therapeutic” and “therapeutically active” are used interchangeably herein, and refer to the prevention or treatment of a disease or condition in a human or other animal, or to the maintenance of good health in a human or other animal, resulting from the administration of a cranberry or grapefruit derivative of the invention, or a composition derived therefrom. Such health benefits can include, for example, nutritional, physiological, mental, and neurological health benefits.

Overview

The present invention is based on the identification of therapeutic cranberry and grapefruit products (e.g., derivatives), and compounds isolated from the products, having novel therapeutic and/or health promoting values (see Tables 1 and 2). In particular, therapeutic cranberry and grapefruit products of the invention are shown herein to exhibit significant anti-cancer and anti-hypercholesterolemia activity when administered to a subject in vivo and when tested in vitro.

In a particular embodiment, therapeutic methods of the invention employ decharacterized cranberry fruit, preferably Tomah presscake, which is enriched for a number of health promoting compounds and exhibits e.g., significant anti-cancer properties when administered to a subject. Moreover, a variety of particular health promoting compounds derived from decharacterized cranberry have been identified and are discussed below.

In another embodiment, therapeutic methods of the invention employ grapefruit products (derivatives) which are novel sources of compounds having significant therapeutic value, in, for example, the prevention or treatment of cancer by, e.g., lowering rates of metastasis. In addition, as described herein, a subset of these grapefruit derivatives are also enriched with compounds suitable for treating disease related to high cholesterol (hypercholesterolemia).

In another embodiment, therapeutic methods of the invention employ compounds derived from cranberry and grapefruit juices which, as shown herein, have anticancer activity (e.g., reduced metastasis rates) e.g., when prepared in concentrated form and administered to a mammal in vivo.

Accordingly, the identification of particular beneficial compounds in cranberry and grapefruit derivatives has allowed for the development of convenient methods and compositions (e.g., formulations) for administering therapeutic compounds to treat or prevent particular diseases. Moreover, the therapeutic compounds and compositions described herein have the additional advantage of being readily manufactured into palatable forms (e.g., as foodstuffs such as juices and food bars or as dietary supplements) for convenient oral administration.

Methods for obtaining and preparing cranberry and grapefruit products of the invention, identifying (e.g., characterizing) and obtaining therapeutic components of the products, evaluating biological activity in vitro and in vivo of the products and components, and methods of using the products and novel compositions containing the products or combinations of components isolated from the products, are discussed in the appropriate subsections below.

TABLE 1
Summary Table of Effectiveness of Different Cranberry Derivatives

| | | anti- | anti-hyper- | |
|---|---|---|---|---|
| | Sample | cancer | cholesterolemic | type of study |
| 1 | cranberry essence (OS2) | − | − | cells |
| 2 | decharacterized cranberry | + | − | animals |
| 3 | cranberry juice, 2x | + | − | animals |

TABLE 2
Summary of Effectiveness of Different Grapefruit Derivatives

| | | anti- | anti-hyper- | |
|---|---|---|---|---|
| | Sample | cancer | cholesterolemic | type of study |
| 4 | grapefruit aroma (OS1) | − | − | cells |
| 5 | grapefruit essence oil | + | + | cells |
| | (OS3) | | | |
| 6 | grapefruit red peel oil | + | + | cells |
| | (OS4) | | | |
| 7 | grapefruit peel processed | − | + | animals |
| 8 | grapefruit juice, pink, 2x | + | − | animals |

Methods for Preparing Cranberry and Grapefruit Derivatives

Cranberry and grapefruit products (i.e., derivatives) of the invention may be isolated from whole cranberry, grapefruits, or juices, peels, rinds thereof, using any suitable art recognized method. Preferred derivatives include cranberry essence, cranberry juice (e.g., concentrated), decharacterized cranberry fruit, grapefruit essence oil, grapefruit peel oil, grapefruit peel (e.g., processed), and grapefruit juice (e.g., concentrated).

In a particular embodiment, decharacterized cranberry fruit is obtained using the method described in U.S. Pat. Nos. 5,320,861 and 5,419,251, hereby incorporated by reference.

In all cases, the cranberry or grapefruit derivatives are preferably obtained in a form suitable for use in a foodstuff, dietary supplement, or pharmaceutical composition. Further, it is understood that with regard to any of the techniques for preparing a cranberry or grapefruit derivative described herein, it may also be desirable to avoid exposing the derivative, or component thereof, to oxygen by, e.g., protective blanketing of the derivative or component with carbon dioxide or nitrogen gas, or by, e.g., exposing the derivative or component, where appropriate, to BHT, ascorbic acid, low temperature, or a combination of these conditions.

Cranberry and Grapefruit Derivatives

As part of the present invention, several cranberry derivatives, including cranberry essence (evaporated juice), concentrated cranberry juice, and decharacterized cranberry fruit were analyzed for health promoting compounds (see Table 2). These cranberry derivatives were analyzed using both chemical analysis and bioactivity assays as described herein. In addition, a number of grapefruit derivatives, including grapefruit aroma, grapefruit essence oil, grapefruit red peel oil, processed grapefruit peel, and grapefruit juice also were studied for their in vitro and in vivo therapeutic activity and analyzed for health promoting compounds (see Table 1).

Accordingly, by way of the studies described herein, it was shown that particular cranberry and grapefruit are novel sources of therapeutically beneficial compounds such as a phenolic acid, flavanoid, pectin, omega-3-fatty acid, tocochromanol, triterpenoid, ellagic (see also Table 15). Decharacterized cranberry, particularly “Tomah presscake”, provides several advantages over currently known sources of such therapeutically beneficial compounds including, for example, a remarkably high concentration of particularly desirable components (e.g., an anthocyanin, a phenolic acid, a proanthocyanidin). Accordingly, the cranberry and grapefruit derivatives of the invention, or components thereof, can be used in foodstuffs, or as dietary supplements or pharmaceutical compositions.

Thus, in one embodiment, the invention provides a cranberry or grapefruit derivative, or a composition comprising one or more components isolated from the cranberry or grapefruit derivative, e.g., see Tables 12-14, which promotes health in a human or other animal. The cranberry or grapefruit products of the invention, and compositions derived therefrom, can additionally contain one or more exogenous (i.e., externally added) compounds to further enhance the therapeutic value of the derivative or composition derived therefrom, for example, by acting in synergism with one or more native components of the derivative. For example, grapefruit derivatives of the invention (and compounds isolated therefrom, such as a flavanone, flavone, liminoid, fiber, essential oil, or glucaric acid) having bioactivity may be used alone or in combination with cranberry derivatives of the invention (such as decharacterized cranberry fruit and compounds isolated therefrom) for the prevention or treatment of a disease or condition in a human or other animal, or for the maintenance of good health in a human or other animal. Such health benefits can include, for example, nutritional, physiological, mental and neurological health benefits.

Phenolic Compounds

In particular, the cranberry and grapefruit derivatives of the invention contain one or more phenolic compounds, such as those listed in Table 13.

Such phenolic compounds can act as potent antioxidants and, therefore, can prevent or delay oxidation reactions which cause various diseases. Accordingly, the cranberry and grapefruit derivatives of the invention and compositions derived therefrom can be used as used as anti-oxidants. For example, they can inhibit lipid peroxidation, scavenge free radicals and active oxygen, inactivate lipoxygenase, and chelate iron ions. They also can be used to inhibit erythrocyte aggregation and sedimentation. Moreover, epidemiological studies have demonstrated that the consumption of phenolic compounds is associated with a reduced risk of cancer.

Accordingly, the cranberry and grapefruit derivatives of the invention and components derived therefrom (e.g., fractions rich in phenolic compounds) can be used to treat cancer or cholesterol-related disorders with fewer side effects compared to standard chemotherapies.

Flavonoids

Cranberry and grapefruit derivatives of the invention also contain one or more flavonoids, such as those listed in Table 14.

Flavonoids have widespread anti-cancer and anti-hypercholesterolemic properties. For example, a flavonoid, in particular, a proanthocyanidin extract, has been found to inhibit a key enzyme associated with cell proliferation and skin cancer (Bosmer et al., Planta Med 62:212-6 (1996)). In addition, the flavonoid quercetin can inhibit tumor production by chemical carcinogens, inactivate some enzymes involved in the metabolism of carcinogens, and inhibit LDL oxidation.

Accordingly, the cranberry and grapefruit derivatives of the invention and components derived therefrom (e.g., fractions rich in flavonoid compounds) can be used to treat cancer and cholesterol-related disorders.

Fiber

Cranberry and grapefruit derivatives of the invention also contain fiber, in particular, pectin, and fiber when administered to animals or humans increases the bulk of the feces and this can relieve, e.g., constipation and may reduce the incidence of diverticula forming in the large intestine of older people. Moreover, there is some evidence that a diet high in fiber can reduce colon cancer and reduce plasma cholesterol concentration.

Accordingly, the cranberry and grapefruit derivatives of the invention and components derived therefrom (e.g., fractions rich in pectin) can be used to treat the above-mentioned disorders.

Fatty Acids

Cranberry and grapefruit derivatives of the invention also contain beneficial fatty acids. Fatty acids, for example, omega-3 fatty acids, are essential for growth and development throughout the life cycle. For example, omega-3 fatty acids are known to play an important role in, 1) the normal function of the retina and brain, especially in new born infants, 2) maintaining favorable serum triglycerides in normal subjects and in patients with hypertriglyceridemia, 3) the normal function of the vascular and neurological systems, and 4) reducing LDL (low density lipoprotein) cholesterol in patients with hyperlipidemia (provided that the saturated fatty acid content in the diet is decreased).

Beneficial fatty acids, also derivable from the derivatives of the invention, play important roles in normal physiological functions including, e.g., overall growth, healthy skin, reproduction, and cardiovascular health.

Accordingly, formulations can be prepared by those of ordinary skill in the art containing a desirable fatty acid derived from a cranberry or grapefruit derivative. Such formulations have application in the medical and pharmaceutical industries for enhancing, maintaining or treating any of the above-mentioned biological functions or disfunctions. In addition, given the wide spectrum of biologic processes affected by these fatty acids, the derivatives of the invention can also be used as a food additive or dietary supplement.

For example, in the food industry, to raise the availability of desirable fatty acids in a consumer's diet, the derivatives of the invention, or compositions derived therefrom (e.g., containing components or fractions thereof), can be added to, for example, juices, bakery products, infant formulas, etc. As dietary supplements, the derivatives of the invention or compositions derived therefrom can be taken in the form of e.g., liquids, pills, or capsules as are known in the art. As discussed further below, methods for formulating such vehicles of administration can be performed using standard techniques.

In another embodiment, the derivatives of the invention or compositions derived therefrom (e.g., containing health-promoting fatty acids) can be fed or otherwise administered to laying hens to produce eggs rich in desirable fatty acids, or to cows or other livestock to produce meat and dairy products rich in such fatty acids. The resultant food products derived from these animals can then be consumed by humans for their enhanced nutritional and health benefits.

Alternatively, the derivatives of the invention or compositions derived therefrom can be fed or otherwise administered to animals, such as pets or domesticated livestock, for therapeutic purposes (e.g., to correct problems such as dry skin, allergic reactions, and cancer).

Tocochromanols

Cranberry derivatives of the invention, in particular, also contain a remarkably high concentration of tocochromanols (a class of compounds that includes tocopherols and tocotrienols), such as α-tocopherol, γ-tocopherol, δ-tocopherol, α-tocotrienol, γ-tocotrienol, δ-tocotrienol, or a combination thereof. A large body of research has shown the importance of tocopherols and tocotrienols in the defense against numerous biological disorders, for example, breast cancer, as described in 60/137,405 by Nawar, W., hereby incorporated by reference.

Tocochromanols are one of the active agents in vitamin E which has been recognized as an anticarcinogenic agent for a number of years (Haenszel et al., Int. J Cancer, 36:43-48 (1985); Menkes et al., N. Engl. J. Med., 315:1250-1204 (1986); Stahelin et al., Ann. NY Acad. Sci., 570:391-399 (1989)). In addition, in vitro and in vivo studies, including human studies, have demonstrated that vitamin E interferes with the development of carcinogenesis that results from exposure to various environmental factors known to enhance oxidant stress (Borek et al., In, Mechanisms of cellular transformation by carcinogenic agents, New York, Pergamon (1987), Borek et al., In, Medical, biochemical and chemical aspects of free radicals, Amsterdam, Elsevier, (1989); Borek et al., Proc. Natl. Acad. Sci. USA 83:1490-1494 (1986); Proc. Natl. Acad. Sci. USA, 88:1953-1957 (1991)). In addition, α-tocopherol, a component of vitamin E, is a hydrophobic, peroxyl radical trapping, chain-breaking antioxidant found in biological membranes. Accordingly, the protective role vitamin E plays in inhibiting a variety of human malignancies is mainly attributed to its components having the ability to protect the lipid material of the organs against oxidation (Ames et al., Science 230:271-279 (1987); Doll et al., J. Natl. Cancer Inst. 66:1193-1194 (1981): Greenwald et al., Cancer 65:1483-1490 (1990); Menzel et al., J. Agr. Food Chem., 20:481-486 (1972)).

Accordingly, the cranberry and grapefruit derivatives of the invention and compositions derived therefrom (e.g., fractions rich in tocochromanols) can be used to treat respiratory, inflammatory, neurological, dermatological, opthalmological, and gastroenterological diseases.

Triterpenoids

In addition, cranberry and grapefruit derivatives of the invention contain one or more triterpenoids, such as, e.g., ursolic acid. Such triterpenoids are known to confer significant health benefits, e.g., as antiinflammatories or hepatoprotectants. For example, ursolic acid is effective in protecting against chemically induced liver injury in laboratory animals and also has antihyperlipidemic properties. Ursolic acid has also been noted for its is antitumor-promotion effects (Liu, J Ethnopharmacol 49:57-68 Is (1995)).

Accordingly, cranberry or grapefruit derivatives of the invention, and components derived therefrom (e.g., fractions rich in triterpenoids), can be used to, e.g., inhibit inflammation, treat liver disorders, and inhibit tumor promotion.

Ellagic Acid

In addition, cranberry and grapefruit derivatives of the invention also contain a plant phenol such as ellagic acid. Plant phenols such as ellagic acid have anticancer properties and ellagic acid in particular, can protect animals against benzo[a]pyrene-induced neoplasia (Lesca, P., Carcinogenesis 4:1651-3(1983)). Accordingly, cranberry or grapefruit derivatives of the invention and components derived therefrom (e.g., fractions rich in ellagic acid) can be used as an anti-cancer agent.

Methods for Isolating, Identifying, and Analyzing Specific Components from Cranberry and Grapefruit Derivatives

To isolate and analyze constituent therapeutic components (compounds) from cranberry and grapefruit derivatives, a variety of art-recognized techniques and assays can be employed. For example, as described in the studies provided herein, oil-based samples (e.g., grapefruit essence oil or grapefruit red peel oil) can be prepared for analysis by converting the fatty acids in the oil to their methyl esters, for example, by refluxing with MeOH/MeO⁻ Na⁺. The resultant methyl esters can then be analyzed, e.g., by gas chromatography.

Phenolic compounds of the cranberry and grapefruit derivatives of the invention can be analyzed and extracted using HPLC analysis and solvent extraction, respectively. The isolated extracts can be dissolved in hexane and then extracted with a methanol/water solution followed by centrifugation. The extract can then be dried, and the residue can be resuspended in methanol/water for HPLC analysis.

For example, tocochromanols contained in an oil fraction derived from a cranberry or grapefruit derivative of the invention can be separated and analyzed using, for example, the methods of Carpenter (Carpenter, Jr., A. P., J. Amer. Oil Chemists' Soc., 56:668 (1979)).

Triterpenoids can be extracted and analyzed using, for example, thin layer chromatography and high-performance liquid chromatography. For example, an the isolated oil fraction derived from a cranberry or grapefruit derivative can be saponified with KOH, the unsaponifiables extracted with ether, and the resultant material can be fractionated on thin-layer chromatography (TLC) plates where the individual bands that are subsequently resolved can be scraped and extracted with a chloroform/methanol solvent. These resultant samples can then be analyzed using, e.g., gas and high-performance liquid chromatography (HPLC).

Other methods known in the art may also be employed, in place of or in combination with, the methods described above for isolating cranberry or grapefruit components, particularly to “scale up” the quantity of the isolated components. For example, chromatographic techniques may be used for isolating components of the cranberry or grapefruit derivatives of the invention, in sufficient and pure quantities, such that the component may be administered alone or as part of a composition or product described herein (e.g., foodstuffs, dietary supplements, pharmaceuticals, etc.). In particular, gas liquid chromatography, gas solid chromatography, high pressure or high performance liquid chromatography (HPLC) (e.g., normal, reverse, or chiral), ion exchange chromatography, or size exclusion chromatography can be employed as described, for example, in Advances in Chromatography, Brown, Eds., Marcel Dekker, Pub. (1998); Basic Gas Chromatography, Harold et al., John Wiley & Sons, Pub. (1997); Column Handbookfor Size Exclusion Chromatography, Wu, Ed., Academic Press, Pub. (1999); Fundamentals of Preparative and Nonlinear Chromatography, Guichon et al., Eds., Academic Press, Pub. (1994); Handbook of Process Chromatography: A Guide to Optimization, Scale-Up and Validation, Hagel et al., Eds., Academic Press, Pub. (1997); HPLC Methods for Pharmaceutical Analysis, Lunn et al., John Wiley & Sons, Pub. (1997); and Practical High-Performance Liquid Chromatography, Meyer, Wiley-Liss, Pub. (1999), each of which is incorporated by reference herein. Such isolated components, which can be separated as “value added” fractions (e.g., fractions having therapeutic value), are typically rich in at least one beneficial component identified from a cranberry or grapefruit derivative described herein. These isolated components or fractions may be further combined to provide a composition rich in more than one component or, e.g., a desired combinations thereof. In addition, a particular formulation intended for the treatment or prevention of a particular disease or condition may be formulated to be rich in those components having a therapeutic effect on the disease or condition (e.g., associated with affecting a change in any of the mechanisms associated with that particular disease or condition). For example, a formulation suitable for administering to a subject with cancer is preferably rich in cranberry or grapefruit derived components having antioxidant and other anti- cancer properties, whereas a formulation for administering to a subject with a dietary need, may be rich in, for example, beneficial fatty acids.

In Vitro Methods for Evaluating the Therapeutic Properties of Cranberry and Grapefruit Derivatives and Components Derived Therefrom

The health promoting properties, e.g., anticancer activity, of cranberry and/or grapefruit derivatives of the invention, and compositions derived therefrom, can be evaluated using a variety of art-recognized cell-based assays, e.g., cell proliferation assays using tumor cells.

For example, in one embodiment, a tumor cell proliferation assay is performed by measuring the incorporation of [³H] thymidine into the DNA of dividing cells, as is known in the art. For example, a solution containing cranberry and/or grapefruit derivatives of the invention, or components derived therefrom (e.g., a phenolic acid rich fraction rich fraction), can be added to tissue culture plates, for example, in decreasing concentrations and incubated at 37° C. for 3 days, after which tritiated thymidine is added to each well to determine the number of dividing cells at each concentration. The cells are further incubated for a sufficient period of time, e.g., 4 hrs, to allow for the incorporation of a detectable radiolabel into the DNA of dividing cells and then medium and excess label are removed. The cells can then be harvested by, e.g., trypsinization, and the amount of radioactivity present in the cells is measured using standard techniques. The concentration at which the cranberry or grapefruit derivatives of the invention exhibit 50% or 90% inhibition of cell growth (respectively, IC50 or IC90) is determined by comparing the radioactivity measured in the extract-treated cells as compared to untreated control cells.

Another method for determining the viability of tumor cells after exposure to an appropriate cranberry and/or grapefruit derivative of the invention, or a component derived therefrom, employs a vital dye (3-[4-5-dimethylthiazol-2-yl)-2,5-diphenyl-tetrazolium bromide (MTT)) that, when mixed into a cell sample, exhibits a detectable signal that distinguishes viable from non-viable cells. The intensity of the blue color, due to a formazan product formed by cellular reduction of MTT by the mitochondrial dehydrogenase of the surviving cells, is then measured as an indication of the viability of the remaining cells (Hansen et al., J. Immunol. Methods, 119:203-210 (1989)). Percent viability can be determined by relating absorbance/concentration of the treated cells to that of the non-treated controls.

To resolve the long term growth effects on cells caused by exposure to an appropriate cranberry and/or grapefruit derivative of the invention, or a component derived therefrom, can be determined by incubating plates containing the cell culture medium plus the MTT reagent at its IC50 concentration at 37° C. Plates are removed at appropriate intervals, the medium aspirated, the cells trypsinized, resuspended, counted with a hemocytometer, and the number of cells plotted against time to construct growth curves.

In Vivo Methods for Evaluating the Therapeutic Properties of Cranberry and Grapefruit Derivatives and Components Derived Therefrom

In another embodiment, cranberry and/or grapefruit derivatives of the invention, and compositions derived therefrom, can be tested for their in vivo therapeutic effect by administering (e.g., orally) the extracts or compositions in a suitable form (e.g., as a food stuff, dietary supplement, or pharmaceutical composition) to a human or other animal, and then observing the physiological effect (e.g., compared to a control). The human or animal can be, for example, suffering from a disease or condition, such as those described herein (e.g., cancer or hypercholesterolemia). Thus, a reduction in the physical symptoms of the disease can be measured as an indication of the therapeutic efficacy of the cranberry and/or grapefruit derivatives or compositions derived therefrom.

In another approach for evaluating anti-tumor activity, cranberry and/or grapefruit derivatives of the invention or compositions derived therefrom (e.g., a fraction thereof) can be used in a controlled animal study where tumors are induced in the animal via diet, by applying a chemical tumor promoter to the skin, or by the implantation of tumor cells in the presence or absence of the test agent (see, e.g., Example 2). Various assays, such as those described below, can then be used to examine the progression of careinogenesis in the presence or absence of the administration of the extracts or compositions of the invention.

Methods of Use

Treatment of Cancer

In one embodiment, cranberry and/or grapefruit derivatives of the invention, and compositions derived therefrom (particularly those containing detectable amounts of, e.g., anthocyanins, phenolics, proanthocyanidins, tocotrienols, or flavonoids), are administered to a mammal (e.g., a human or animal) to treat or prevent cancer. Such derivatives and compositions also can be administered in combination with other anti-cancer agents. In particular, the cranberry and grapefruit derivatives of the invention, and compositions derived therefrom, can be administered separately or together and/or with either tamoxifen and/or a flavonoid for the treatment of, for example, breast cancer. These combinations of agents encompassed by the invention are particularly effective because of the ability of tocochromanols to act in synergy with tamoxifen and/or flavonoids in the inhibition of tumorigenic cells.

For example, it is known that most breast cancers consist of hormone-dependent as well as hormone independent cells. The drug tamoxifen, a synthetic non-steroidal estrogen antagonist, has been widely used in the treatment of hormone-responsive breast cancer. In addition, the inhibitory effects of various combinations of the palm oil tocotrienol-rich fractions as well as individual tocotrienols in combination with tamoxifen on at least two breast cancer cell lines (i.e., estrogen receptor-negative MDA-MB-435 and estrogen receptor-positive MCF-7) have been demonstrated (Guthrie, et al., Asia Pacific J. Clin. Nutr. 41-45 (1997)).

Prior to the present invention, treatment of cancer patients with tamoxifen had several drawbacks. For example, tumors can develop resistance to tamoxifen, possibly caused by the drug's intrinsic estrogen antagonist properties (Osborne et al., J. Natl. Cancer Inst. 87:746-750 (1995)). Also, tamoxifen may increase the incidence of new primary malignancies, e.g. endometrial, liver, and colorectal cancers (Rutgrist et al., 1995). Accordingly, the present invention provides the advantage of enabling the administration of tamoxifen in lower doses, for example, in combination with a cranberry derivative of the invention or a composition derived therefrom (particularly one having a high tocotrienol content) to avoid these undesirable effects.

Treatment of High Cholesterol and Diseases Arising Therefrom

In another embodiment, the cranberry and grapefruit derivatives of the invention, and compositions derived therefrom (e.g., those having high tocochromanol and/or phytochemical content), can be used to treat or prevent high cholesterol and related diseases such as arteriosclerosis and heart disease. Indeed, the efficacy of various tocochromanol and plant derived phytochemicals in reducing cholesterol levels in animals, including humans, is well supported in the scientific literature.

Animal studies have shown that tocotrienol-containing bran oil, barley oil, and palm oil suppressed cholesterologenesis when fed to chicken. In addition, hypercholesterolemic pigs fed the TRF-supplemented diet showed a 44% decrease in total serum cholesterol and a 60% decrease in LDL cholesterol, with the decrease persisting for 8 weeks even after putting the animals back on the control corn-based diet (Quereshi et al., In, International Palm Oil Conference, pp 45-47 (1988)).

Similarly, a study involving 47 hypercholesterolemic subjects administered dietary supplements containing 200 mg of TRF per day for 4 weeks resulted in, respectively, a 15-22% and 10-20% reduction in serum total and LDL cholesterol (Quereshi, et al., Lipids 20:817-824 (1985); Quereshi, et al., Am. J. Clin. Nutr., 53:1021S-1026S (1991)). In addition, in studies where the hypocholesterolemic effect of tocotrienols was compared with that of other drugs, the tocotrienols were more effective. For example, in a study involving chickens, T3 was demonstrated as being twice as effective as Lovastatin™, a drug currently used for cholesterol control in humans. And most of the drugs most commonly used today in the therapy of hypercholesterolemia (i.e., Nicotinic acid (Grundy et al., J. Lipid Res., 22:24-36 (1981)), Compactin™ and Lovastatin™ (Illingworth et al., Eur. Heart J., Supp. E: 103-111 (1987); Endo et al., Biotechnology, 26:301-320 (1994)), Cholestyramine™ and Colestipol™ (Shepherd et al., Biochem. Soc. Trans. 15:199-201 (1980)), Clofibrate™ and Gemfibrosil™ (Kesaniemi et al., JAMA, 251:2241-2246 (1984)) and Probucolm are known to produce various side effects (Illingworth et al., Am. J. Cardiol., 60:33G-42G (1987)). In contrast, no toxic effects were observed in the studies where tocotrienols were administered.

Accordingly, the cranberry and grapefruit derivatives of the invention, and compositions derived therefrom, can be used in the treatment of high cholesterol (cholesterolemia) and other associated conditions such as heart disease.

Treatment of Other Diseases and Disorders

In yet another embodiment, the cranberry and grapefruit derivatives of the invention and compositions derived therefrom (particularly those having high, e.g., a high tocochromanol content) can be used in the treatment or prevention of a wide range of other diseases and disorders that include aging, respiratory, inflammatory, neurological, dermatological, opthalmological, and gastroenterological diseases. Indeed, a large volume of reported research provides evidence that vitamin E-containing tocochromanols plays a critical role in the above-mentioned conditions. In addition, a number of studies indicate that citrus-derived flavanones, flavones, liminoids, fiber (e.g., pectin), essential oils, and glucaric acid have, e.g., anticancer-activity and/or anti-hypercholesterolemic activity (for a review, see Johns et al., Recent Advances in Phytochemistry, pp.31-52, Plenum Press (1997).

For example, cranberry or grapefruit derivatives of the invention and compositions derived therefrom can be used to prevent endothelial injury, such as ischemic and reperfused myocardium and ulcers. In addition, the extracts and compositions can be used to inhibit tumor necrosis factor biosynthesis that, in turn, decreases inflammation (e.g., by inhibiting respiratory bursts of neutrophils or via free radical scavenging). Accordingly, cranberry or grapefruit derivatives of the invention and compositions derived therefrom (particularly those having high tocochromanol and/or flavanoid content) can be used as anti-inflammatory agents for the prevention and treatment of a wide variety of diseases and conditions involving minor, acute and chronic inflammation.

Cranberry or grapefruit derivatives of the invention and compositions derived therefrom (particularly those having high tocochromanol (particularly those having high tocochromanol and/or flavanoid content) also can be used to treat glucose intolerance in diabetes mellitus, and/or to restore acute glucose-induced insulin response in non- insulin-dependent diabetes mellitus.

In addition to the above-stated uses, cranberry or grapefruit derivatives of the invention and compositions derived therefrom (particularly those having high tocochromanol and/or flavanoid content) can be used to enhance the immune response in animals and humans, for example, by reducing the amount of fatty acids in biological tissues. Since fatty acid levels effect the immune system, the compounds of this invention may serve as immunoregulators. They may, for example, be used to increase antibody titers to foreign proteins.

In addition, the reduction in fatty acid, cholesterol, fatty acid and/or glucose levels induced by the compounds of the invention can be obtained without attendant substantial weight loss, resulting in an increased feed to protein conversion ratio. Therefore, the extracts and compositions of the invention can be used to increase feed conversion efficiency.

Hypercholesterolemic diseases and conditions that can be treated using the cranberry or grapefruit derivatives of the invention and compositions derived therefrom include, but are not limited to, atherosclerosis, arteriosclerosis, xanthomatosis, hyperlipoproteinemias, and familial hypercholesterolemia.

Thrombotic diseases and conditions that may be treated using cranberry or grapefruit derivatives of the invention and compositions derived therefrom include, but are not limited to, pulmonary disease (for example, involving reduced conductance, compliance, or constriction), excessive fluid accumulation or pulmonary edema, respiratory distress, asthma, pulmonary vascular permeability, pulmonary vasoconstriction, pulmonary hypertension, pulmonary embolism, cardiac ischemia, myocardial infarction, cardiopulmonary bypass associated dysfunction, vasoconstriction, organ dysfunction, platelet dysfunction, cardiac disease, chronic obstructive arterial disease caused by arteriosclerosis, vasoconstriction, renal artery stenosis, myocardial infarction, stroke, deep vein thrombosis, peripheral arterial occlusion, and other blood system thromboses.

The antioxidizing properties of the cranberry or grapefruit derivatives of the invention and compositions derived therefrom may also be applied to, but are not limited to, the treating and preventing of cancerous conditions by, for example, preventing or limiting cancer-causing mutations in the genetic material of an animal or a human.

Antiatherogenic diseases and conditions that can be treated using cranberry or grapefruit derivatives of the invention and compositions derived therefrom include, but are not limited to, atherosclerosis, arteriosclerosis, myocardial infarction, ischemia (i.e., myocardial ischemia, brain ischemia, and renal ischemia) and strokes.

Inflammatory diseases and conditions that can be treated using cranberry or grapefruit derivatives of the invention and compositions derived therefrom include, but are not limited to, essential hypertension, hypertension of congestive heart failure, renal dysfunction caused by reduced myocardia output, endotoxemia, chronic liver disease or hypertension, pulmonary inflammation in asthma, lung injury (bronchitis, pneumonia, or acute); rheumatic diseases (for example, rheumatoid arthritis or systemic lupus erythematosus), inflammatory bowel disease (for example, ulcerative colitis), irritable bowel disease (such as villous adenoma), gastrointestinal disorders caused by excess acids, pepsin or bile salts, Zollinger-Ellison syndrome, skin diseases or trauma (such as bums or acid or caustic injury), gout, Bartter's syndrome, fever, rheumatoid diseases, pain, and functio laesa.

Immunoregulatory diseases and diseases that can be treated using cranberry or grapefruit derivatives of the invention and compositions derived therefrom include, but are not limited to, autoimmune diseases, for example, AIDS, chronic fatigue syndrome, graft rejections, and other viral diseases that impair the immune system.

Synergy with Other Components Derived From Cranberry or Grapefruit Derivatives and/or Exogenous Compounds

In another embodiment, cranberry and/or grapefruit derivatives of the invention, or one or a combination of components derived therefrom, are administered to a subject with an additional (exogenous) compound, e.g., an anti-cancer such as tamoxifen, and/or in combination with a flavonoid for the treatment or prevention of cancer. These combinations of agents encompassed by the invention are particularly effective because of their known ability to act in synergy in the inhibition of tumorigenic cells. The flavonoid may be contained in or derived from cranberry or citrus fruit such as, e.g., a grapefruit.

Flavonoids are polyphenolic compounds which occur in plant foods, particularly citrus. These compounds include the flavones, e.g tangeretin; the flavanones, e.g. hesperetin; the isoflavones, e.g genistein; and the flavonols, e.g. quercetin and these are predicted to be present at various levels in one or more of the grapefruit derivatives of the invention. It is understood that with no more that routine experimentation in combination with the teachings of the specification the skilled artisan would be able to determine which cranberry or grapefruit derivatives of the invention have desirable levels of a polyphenolic compounds suitable for administering to a mammal alone or in combination with another derivative of the invention or exogenous compound. Several studies have demonstrated the anticancer properties of flavonoids from various plant sources (Cook et al., J. Nutr. Biochem. 7:66-76 (1996); Hertog et al., Nutr. Cancer 20:21-29 (1993); Middeleton et al., Trends Pharm. Sci., 5:335-338 (1984)). Further, various combinations of flavonoids from different sources have been shown to be synergistic in their ability to inhibit the proliferation of a breast cancer cell line (MDA-MB-435 cells).

In particular, synergistic effects between the tocochromanols and flavonoids, with γ-T₃ and tangeretin being the most effective combination, have been observed when tested for their ability to inhibit growth in MDA-MB-435 and MCF-7 breast cancer cells (IC₅₀ 0.05 μg/mL and 0.02 μg/mL, respectively) (Guthrie et al., Asia Pacific J. Clin. Nutr. 6:41-45 (1997)). In addition, with few exceptions, combinations (1:1: 1) of tocotrienols, flavonoids, and tamoxifen were more effective than 1:1 combinations of T₃ and flavonoids, T₃ and tamoxifen, or flavonoids, and tamoxifen. The most potent combinations are γ-T₃/tangeretin/tamoxifen with the MDA-MB-435 cells and δ-T₃ /hesperetin/tamoxifen with the MCF-7 cells.

Accordingly, cranberry and grapefruit derivatives of the invention and compositions derived therefrom (particularly those having high tocochromanol and/or flavonoid content) can be used alone or in combination with tamoxifen and/or flavonoids as potent anti-cancer agents.

Formulations and Methods of Administration

The cranberry and grapefruit derivatives of the invention and components derived therefrom can be administered to a subject in any suitable form. For example, the extracts and compositions of the invention are sufficiently stable such that they can be readily prepared in a form suitable for adding to various foodstuffs including, for example, juice, fruit drinks, carbonated beverages, breakfast cereals, biscuits, cakes, muffins, cookies, toppings, bread, bagels, fiber bars, soups, crackers, baby formulae, salad dressings, cooking oils, and meat extenders.

In addition, the cranberry and grapefruit derivatives of the invention and compositions derived therefrom can be formulated as a pharmaceutical composition (e.g., a medicinal drug) for the treatment of specific disorders.

In another embodiment, the cranberry and grapefruit derivatives of the invention and compositions derived therefrom can be formulated as a dietary supplement.

Suitable additives, carriers, and methods for preparing such formulations are well known in the art.

For example, pharmaceutical compositions may take the form of tablets, capsules, emulsions, suspensions and powders for oral administration, sterile solutions or emulsions for parenteral administration, sterile solutions for intravenous administration and gels, lotions and cremes for topical application. The pharmaceutical compositions may be administered to humans and animals in a safe and pharmaceutically effective amount to elicit any of the desired results indicated for the compounds and mixtures described herein. In addition, the extracts of the invention may be used in cosmetics.

The pharmaceutical compositions of this invention typically comprise a pharmaceutically effective amount of a cranberry and/or grapefruit derivative, or fraction thereof, containing, for example, a phenolic acid, flavonoid, pectin, omega-3-fatty acid, tocochromanol, triterpenoid, ellagic acid, or combination thereof (as pertains to cranberry, see also Table 15), and if suitable a pharmaceutically acceptable carrier. Such carriers may be solid or liquid, such as, for example, cornstarch, lactose, sucrose, olive oil, or sesame oil. If a solid carrier is used, the dosage forms may be tablets, capsules or lozenges. Liquid dosage forms include soft gelatin capsules, syrup or liquid suspension.

Therapeutic and prophylactic methods of this invention comprise the step of treating patients or animals in a pharmaceutically acceptable manner with the compositions and mixtures described herein. As used herein, the term “therapeutically effective amount” refers to an amount effective to achieve a desired therapeutic effect, such as, achieving desirable levels of anti-cancer activity (e.g., reduction of tumor size, incidence of metastasis) or anti-cholesterol activity (e.g., lower blood levels of LDL-cholesterol and total serum cholesterol and higher ratios of HDL-cholesterol to LDL-cholesterol, reduced apoB secretion). The pharmaceutical compositions of this invention may be employed in a conventional manner for the treatment and prevention of any of the aforementioned diseases and conditions. Such methods of treatment and prophylaxis are well-recognized in the art and may be chosen by those of ordinary skill in the art from the available methods and techniques. Generally, dosage ranges may be from about 1 to about 1000 mg/day. However, lower or higher dosages may be employed. The specific dosage and treatment regimens selected will depend upon factors such as the patient's or animal's health, and the severity and course of the patient's (or animal's) condition and the judgment of the treating physician.

The cranberry and grapefruit derivatives of the invention and compositions derived therefrom also can be used in combination with conventional therapeutics used in the treatment or prophylaxis of any of the aforementioned diseases. Such combination therapies advantageously utilize lower dosages of those conventional therapeutics, thus avoiding possible toxicity incurred when those agents are used alone.

In foodstuffs, the cranberry and grapefruit derivatives of the invention and compositions derived therefrom can be used with any suitable carrier or edible additive. For example, the cranberry and grapefruit derivatives of the invention may be used in a variety of foodstuffs, such as drinks, for example, juice drinks, sports drinks, and drink mixes. Advantageously, the above-mentioned foodstuffs may be included in low fat, low cholesterol, or otherwise restricted dietary regimens.

Pharmaceutical compositions, dietary supplements, and foodstuffs of the present invention can be administered to humans and animals such as, for example, livestock and poultry. Once an animal has consumed or otherwise been administered the composition, it can advantageously retain the hypocholesterolemic, anti-cancer, or other advantageous biological activities of the administered compounds. Accordingly, an animal raised under these conditions, or any product derived therefrom, such as, for example, milk, may be consumed by a human or another animal to derive the benefits of the derivatives of the invention or compositions derived therefrom. For example, a chicken which ingests feed fortified with the derivatives of the invention may later be eaten by a human to derive the cholesterol-reducing benefits.

This invention is further illustrated by the following examples which should not be construed as limiting.

### EXAMPLE 1

### In Vitro Demonstration of the Anti-cancer Properties of Cranberry and Grapefruit Derivatives

The following studies were performed to examine the anti-cancer properties of cranberry and grapefruit derivatives.

Four different cranberry and grapefruit extracts were prepared using methods described herein and tested for their ability to inhibit the growth of two different human breast cancer cell lines (i.e., MDA-MB-435 and MCF-7). In each case, both grapefruit essence oil (OS3) and grapefruit red peel oil (OS4) extracts of the invention demonstrated the ability to inhibit the growth of each tumor cell line with greater growth inhibition being seen against the estrogen receptor positive cell line MCF-7.

In particular, grapefruit aroma (OS 1), cranberry essence (OS2), grapefruit essence oil (OS3), and grapefruit red peel oil (OS4) extracts were prepared using methods described herein and by partially evaporating under nitrogen gas to remove any traces of methanol which may be to be toxic to cells. The physical characteristics of each particular extract after partial evaporation are presented in Table 3. The extracts OS1 and OS2 were soluble in DMSO, but not in water, 95% ethanol, 70% ethanol or methanol. The extracts OS3 and OS4 were soluble in 95% ethanol, but not 70% ethanol, methanol, DMSO, or water. For conducting the following experiments, stock solutions of each extract were made in DMSO (OS1 and OS2) or in 95% ethanol (OS3 and OS4).

TABLE 3
Physical characteristics of four different
grapefruit and cranberry extracts.

| | | | Density after |
|---|---|---|---|
| | | % weight loss after | evaporation |
| Extract | Origin | evaporation | (g/mL) |
| OS1 | Grapefruit aroma | 32.43 | 0.9426 |
| OS2 | Cranberry essence | 34.37 | 0.9820 |
| OS3 | Grapefruit essence oil | 24.33 | 0.8382 |
| OS4 | Grapefruit red peel oil | 29.21 | 0.8450 |

The in vitro assay was performed as follows. First, the human breast cancer cell lines MDA-MB435 (estrogen receptor-negative) and MCF-7 (estrogen receptor-positive) were cultured under standard conditions using, minimum essential medium (alpha modification, 3.7 gm of sodium bicarbonate per liter, 10% v/v fetal calf serum). Media for culturing MCF-7 cells was further supplemented with 1 mM sodium pyruvate, 10 ug/mL insulin, 1% v/v fungizone (antibiotic/antimycotic, 10,000 units/mL penicillin G sodium, 10,000 ug/mL streptomycin sulfate and 25 ug/mL amphotericin B in 0.85% saline)).

Next, cells were plated at a density of 2×10⁴ cells/well in 96-well, flat-bottomed tissue culture plates in a total volume of 200 uL of medium and incubated at 37° C., with or without grapefruit aroma (OS 1), cranberry essence (OS2), grapefruit essence oil (OS 3), or grapefruit red peel oil (OS4) extracts. The plates were incubated for 48 hours at 37° C. and [3H] thymidine was then added to determine the number of dividing cells at each concentration of sample. The cells were reincubated for 4 hours, after which the medium and excess radiolabel were removed and cells were harvested and assayed for incorporated radioactivity as a measure of cell proliferation. Accordingly, the percentage of dividing cells was determined by comparing the number of disintegrations per minute of the treated cells (average of 3 wells/concentration) with that obtained for the control cells. The concentrations at which 50% and 90% growth inhibition occurred was determined as the IC50 and IC90 values for each extract. Results are presented in Tables 4-5 and represent the average of 3 experiments ±SEM.

TABLE 4
The effect of grapefruit aroma, cranberry essence, grapefruit essence oil,
and grapefruit red peel oil on the proliferation of MDA-MB-435 estrogen
receptor-negative human breast cancer cells in culture.

| | | IC50 | IC90 |
|---|---|---|---|
| Extract | Origin | (μg/mL) | (μg/mL) |
| OS1 | Grapefruit aroma | >4000 | >4000 |
| OS2 | Cranberry essence | >4000 | >4000 |
| OS3 | Grapefruit essence oil | 32.9 ± 5.2 | 37.0 ± 3 |
| OS4 | Grapefruit red peel oil | 33.1 ± 2.5 | 40.0 ± 3 |

TABLE 5
The effect of grapefruit aroma, cranberry essence, grapefruit essence oil,
and grapefruit red peel oil on the proliferation of MCF-7 estrogen
receptor-positive human breast cancer cells in culture.

| | | IC50 | IC90 |
|---|---|---|---|
| Extract | Origin | (μg/mL) | (μg/mL) |
| OS1 | Grapefruit aroma | >4000 | >4000 |
| OS2 | Cranberry essence | >4000 | >4000 |
| OS3 | Grapefruit essence oil | 24.1 ± 1.9 | 32.0 ± 4 |
| OS4 | Grapefruit red peel oil | 21.0 ± 2.4 | 31.0 ± 1 |

In summary, grapefruit aroma and cranberry essence displayed no cytotoxicity against either MCF-7 (estrogen receptor positive) or MDA-MB-435 (estrogen receptor negative) human breast cancer cell lines at concentrations up to 4 mg/ml (the compounds were not tested at concentrations any higher than this due to the constraints of solubility and toxicity due to the vehicle (DMSO)). By contrast, both grapefruit essence oil and red peel oil showed high levels of anti-cancer activity against both cell lines. In particular, the dose response was extremely steep between the IC50 and IC90 values. In addition, at concentrations within 5 to 10 μg/ml above the IC90 values, almost all of the cancerous cells were completely eliminated after incubation with grapefruit essence extract (OS3) or grapefruit red peel oil extract (OS4). These data indicate that these breast cancer cell lines are very susceptible to the type of damage inflicted by these particular extracts (or components therein), and that once a threshold amount of damage is induced, the cells undergo apoptosis.

### EXAMPLE 2

### In Vivo Demonstration of the Anti-cancer Properties of Cranberry and Grapefruit Derivatives

The following studies were performed to examine the anti-cancer properties of cranberry and grapefruit derivatives when administered to a mammal.

Four different cranberry and grapefruit extracts were prepared using methods described herein and tested for their anti-cancer activity when administered to a test animal having a cancer. In particular, processed grapefruit peel, concentrated pink grapefruit juice, decharacterized cranberry, and concentrated cranberry juice were tested. At least decharacterized cranberry and both the concentrated cranberry juice and concentrated pink grapefruit juice demonstrated the ability to reduce the incidence of tumor metastasis in a test animal with a cancer. This was taken as an indication that certain cranberry and grapefruit derivatives of the invention (and thus, components thereof) have powerful anticancer properties when administered to a mammal.

The in vivo assay for testing the anticancer properties of the cranberry and grapefruit derivatives of the invention was performed as follows. First, 24 female athymic nude mice (NCR-nu/nu), aged 3 weeks, were assigned to 1 of 5 experimental groups and housed under standard conditions. Each group of test animals was then put on either a control diet or test diet comprising a cranberry or grapefruit derivative. Each of the diets contained 5% corn oil (wt/wt) and the amount of dextrose was adjusted to allow for the sugar content of a particular juice being administered. The five groups of test animals were given the following treatments: group 1, control; group 2, concentrated cranberry juice; group 3, concentrated pink grapefruit juice; group 4, decharacterized cranberry (5%); and group 5, processed grapefruit peel (5%); After spending one week on the foregoing diets, the test animals were inoculated with cells from the estrogen receptor-negative MDA-MB-435 human breast cancer cell line (1×10⁶ cells suspended in 50 μL of phosphate-buffered saline (PBS)).

To carry out this procedure of experimentally seeding the test animal with a tumorigenic cell line, mice were anesthetized with metofane, and the tumor cells were injected into a right-sided mammary fat pad that had been exposed by a small incision. The mice were weighed and the inoculation site and auxiliary lymph nodes palpated at weekly intervals. When primary tumors became palpable, the maximum length and width of each were measured with calipers weekly until completion of the study and corresponding surface areas were calculated. The mice were the sacrificed at 11 weeks post injection of the tumor cells.

At necropsy, body weights and primary tumor weights were recorded and the number of macroscopic lung metastases was assessed using standard techniques. In addition, the auxiliary lymph nodes and lungs were fixed in 10% neutral buffered formalin, embedded in paraffin, sectioned, stained with hematoxylin and eosin, and examined by light microscopy to determine the presence of metastases in these organs. The statistically analysis of differences in the incidence of primary tumors and metastases were evaluated using the chi-square test. Other statistical comparisons were made by Student's unpaired t test with p<0.05 being considered statistically significant.

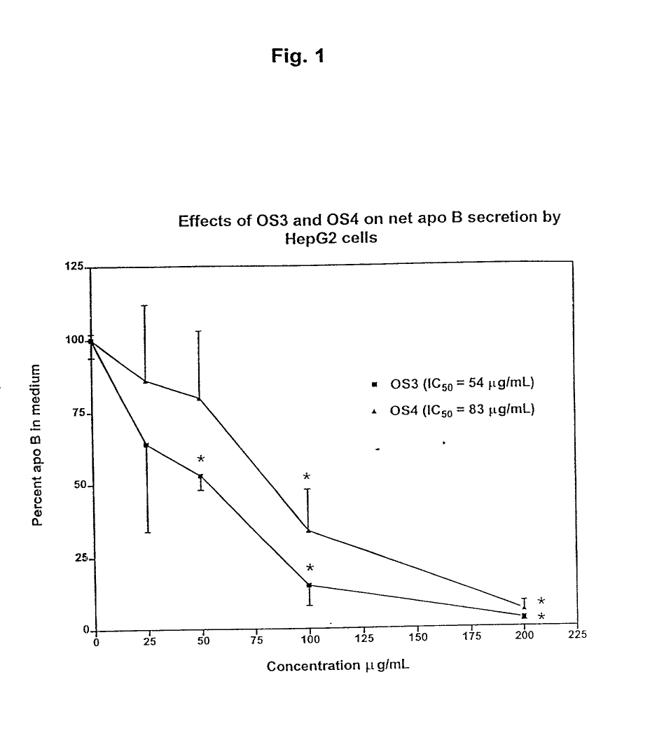

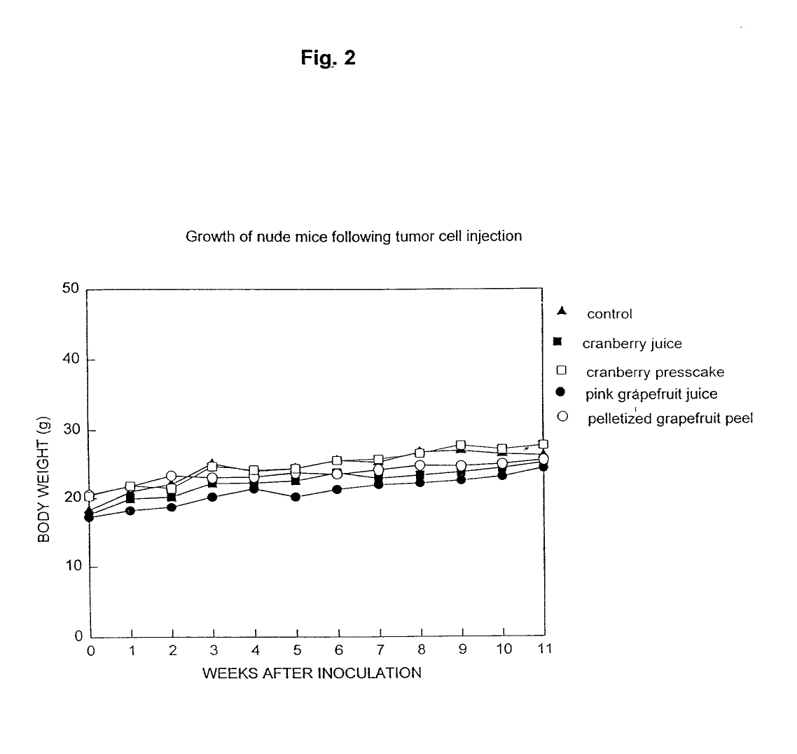

In determining if any of the cranberry of grapefruit based diets influenced cancer growth, the overall body weight and growth of mammary fat pad tumors was assessed. The growth rate of the animals was determined to be similar in all of the test groups (FIG. 1). Next, a comparison of the cumulative incidence of mammary fat pad tumors in the different test groups, over the 11-week experimental period, was performed as shown in FIG. 2. In groups given concentrated cranberry juice, concentrated pink grapefruit juice, or decharacterized cranberry, the incidence of mammary fat pad tumors was significantly reduced (p<0.01). Processed grapefruit peel appeared to have little or no effect on tumor incidence under these conditions. Decharacterized cranberry delayed the onset of tumors by 4 weeks whereas concentrated cranberry juice delayed the onset of tumors by 2 weeks.

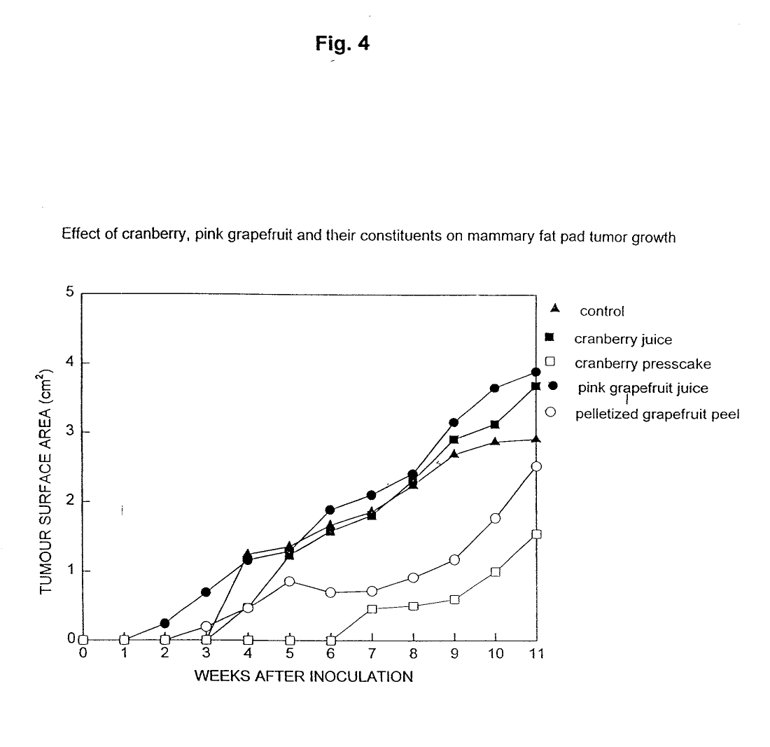

The final incidence of mammary fat pad tumors in each dietary group was 84.7% for the control, 78.6% for the test animals administered processed grapefruit peel, 39.4% for the test animals administered concentrated cranberry juice, 38.9% for the test animals administered concentrated pink grapefruit juice, and 32.9% for the test animals administered decharacterized cranberry. In addition, mammary fat pad tumor size was inhibited most significantly in test animals administered decharacterized cranberry (FIG. 4). The differences between the control and the experimental groups were statistically significant except for the group given processed grapefruit peel (p<0.01).

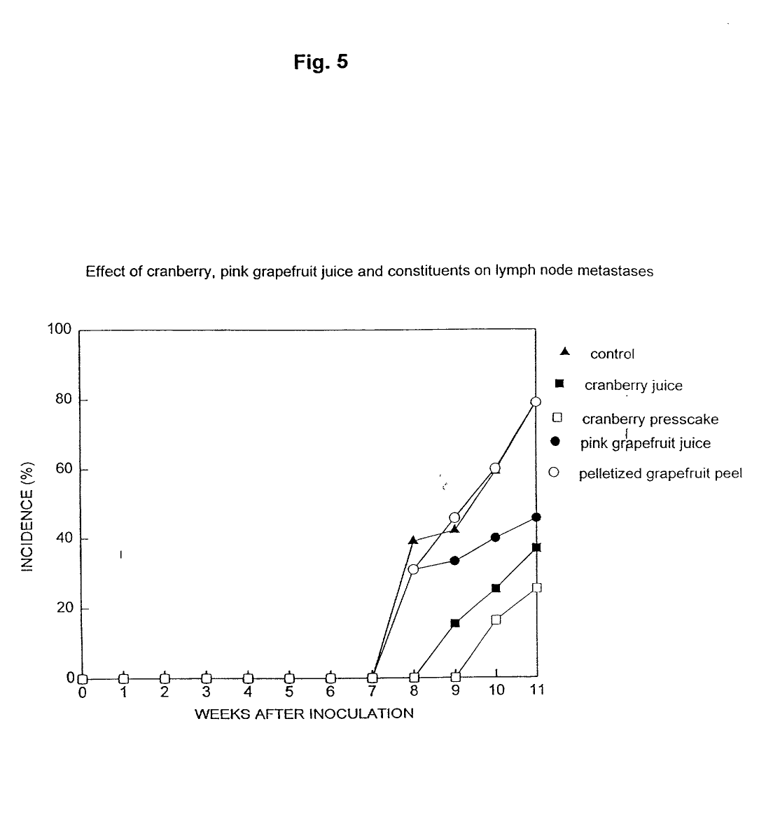

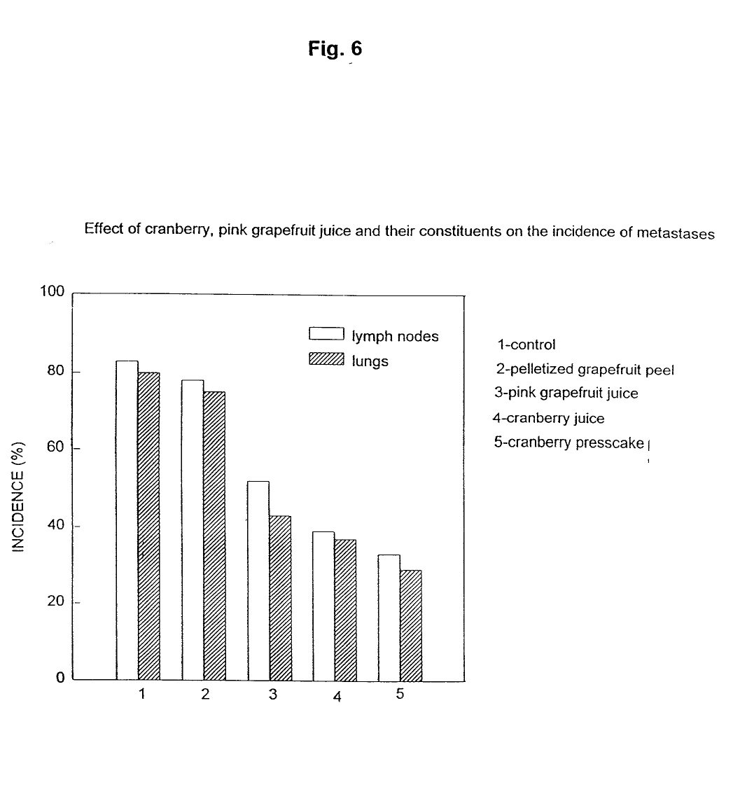

To further characterize the anticancer properties of the cranberry and grapefruit derivatives of the invention, the occurrence of lymph node and lung metastases were evaluated in test animals inoculated with a tumorigenic cell line and administered one of the above cranberry or grapefruit based test diets. The effects of administering concentrated cranberry juice, concentrated pink grapefruit juice, decharacterized cranberry, or processed grapefruit peel on lymph node metastases in a test animal are shown in FIGS. 5-6. Importantly, metastases did not appear in any of the test animals administered a cranberry or grapefruit derivative until week 8. In addition, decharacterized cranberry markedly reduced the incidence of lymph node metastases when compared to the control, followed by concentrated cranberry juice and concentrated pink grapefruit juice (p<0.01). No difference was observed between the group given processed grapefruit peel and control.

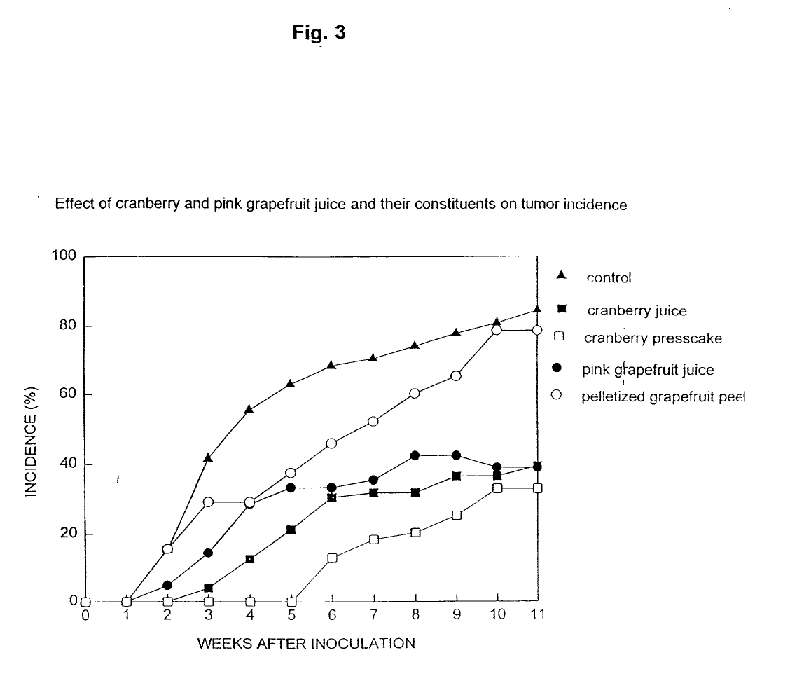

As yet another determination of the anticancer properties of the cranberry and grapefruit derivatives of the invention, the occurrence of macroscopically detectable lung metastases in the above test animals was also assessed (see Table 6 and FIG. 3). The incidence of lung metastases was significantly reduced in test animals given decharacterized cranberry followed by either concentrated cranberry juice or concentrated grapefruit juice, as compared to control animals (p <0.01). There was no statistically significant difference between the control and the group given processed grapefruit peel.

TABLE 6
Effect of cranberry juice, pink grapefruit juice,
decharacterized cranberry, and processed grapefruit peel
on the incidence of microscopic lung metastases.

| | INCIDENCE | MEAN NO. |
|---|---|---|
| GROUP | (%) | PER MOUSE |
| control | 83 | 4.3 ± 0.9 |
| processed grapefruit peel | 75 | 4.2 ± 0.7 |
| conc. pink grapefruit juice | 43 | 1.7 ± 0.3 |
| conc. cranberry juice | 37 | 1.2 ± 0.2 |
| decharacterized cranberry | 29 | 0.6 ± 0.1 |

Accordingly, it was concluded that certain cranberry and grapefruit derivatives (and thus components thereof) have powerful anticancer properties and, for example, when administered to a mammal, can substantially improve the outcome of a mammal having a cancer by lowering tumor growth and rates of metastasis.

### EXAMPLE 3

### In Vitro Demonstration of the Cholesterol Lowering Properties of Cranberry and Grapefruit Derivatives

The following studies were performed to examine the cholesterol lowering properties of cranberry and grapefruit derivatives.

Four different cranberry and grapefruit extracts were prepared using methods described herein and tested for their cholesterol-lowering potential using a human liver cell line (HepG2). In particular, grapefruit aroma (OS 1), cranberry essence (OS2), grapefruit essence oil (OS3), and grapefruit red peel oil (OS4) extracts were tested. At least two of the tested extracts, grapefruit essence oil and grapefruit red peel oil demonstrated the ability to reduce the amount of apolipoprotein B (apoB) secreted from the human liver cells. This was taken as a indication that these extracts of the invention are capable of causing beneficial changes in liver function relating to cholesterol metabolism.

The human liver cells (i.e., hepatoma HepG2 cells) of this example are known to secrete and catabolize lipoproteins similar to LDL and have been used as a model of human liver function relating to cholesterol metabolism. Thus, the ability the grapefruit aroma (OS 1), cranberry essence (OS2), grapefruit essence oil (OS3), and grapefruit red peel oil (OS4) extracts of the invention to change HepG2 secretion of lipoproteins was assayed in order to determine if the extracts of the invention have cholesterol lowering potential.

The assay was performed as follows. First, HepG2 cells were cultured in minimum essential medium (supplemented with 10% fetal bovine serum or 1% bovine serum prior to experimentation) and co-cultivated with a negative control extract (carrier liquid at the same concentration) or increasing concentrations (25-200 μg/mL) of cranberry or grapefruit extracts made up in the carrier liquid. After 24 hours of exposure to the extracts, the cell media was assayed for the presence of apoB using an enzyme-linked immunosorbent assay (ELISA). In particular, cells were washed and dissolved in 0.1 N NaOH for protein determination and the apo B content of the medium was calculated in μg per mg of cell protein and expressed as percent of control (medium of cells incubated with carrier liquid) and these results are presented in Table 7 and in FIG. 1.

The results show that increasing concentrations of either grapefruit essence oil (OS3) or grapefruit red peel oil (OS4) extracts caused a dose-dependent reduction of apo B in the cell medium. The highest non-toxic dose of either grapefruit essence oil extract (OS3) or grapefruit red peel oil extracts (OS4) (200 μg/mL) significantly lowered medium apo B by 97% and 94%, respectively. A significant apo B reduction was also produced by grapefruit essence oil extracts (OS3) at concentrations 100 and 50 μg/mL, respectively, (85% and 47%) and by grapefruit red peel oil extract (OS4) at the concentration 100 μg/mL (66%). The concentrations required for 50% reduction of medium apo B (or IC₅₀ values) were 56 μg/mL for grapefruit essence oil extract (OS3) and 70, μg/mL for grapefruit red peel oil extract (OS4). In contrast, neither the grapefruit aroma extract (OS 1) nor the cranberry essence extract (OS2) did not significantly affect levels of apo B in the medium at any of the concentrations tested.

TABLE 7
Changes in overall apo B production in HepG2 cells exposed to increasing
concentrations of grapefruit extracts.

| | | Conc. | % apo B |
|---|---|---|---|
| Extract | N | μg/mL | in medium |
| grapefruit aroma (OS1) | 4 | 0 | 100 ± 20 |
| | 4 | 25 | 102 ± 15 |
| | 4 | 50 | 93 ± 23 |
| | 4 | 100 | 90 ± 22 |
| | 4 | 200 | 88 ± 30 |
| grapefruit essence oil (OS2) | 4 | 0 | 100 ± 22 |
| | 4 | 25 | 85 ± 15 |
| | 4 | 50 | 93 ± 30 |
| | 4 | 100 | 100 ± 4 |
| | 4 | 200 | 96 ± 11 |
| grapefruit essence oil (OS3) | 4 | 0 | 100 ± 6 |
| | 4 | 25 | 64 ± 30 |
| | 4 | 50 | 53 ± 5* |
| | 4 | 100 | 15 ± 7* |
| | 4 | 200 | 3 ± 1* |
| grapefruit red peel oil (OS4) | 4 | 0 | 100 ± 2 |
| | 4 | 25 | 86 ± 26 |
| | 4 | 50 | 80 ± 23 |
| | 4 | 100 | 34 ± 14* |
| | 4 | 200 | 6 ± 3* |

Means ± SEM. *significantly different from control, p < 0.01.

Thus, these results show that at least two out of the four cranberry and grapefruit derivatives tested, i.e., grapefruit essence oil (OS3) and grapefruit red peel oil (OS4), have cholesterol-lowering potential when tested using human liver cells (HepG2). The grapefruit essence appeared to have more potent apo B-lowering potential than the grapefruit red peel oil. Moreover, using a MTT assay to assess cell viability, it was determined that none of the cranberry of grapefruit derivatives at the dosages tested were toxic to cells. Accordingly, these extracts are healthy sources of beneficial compounds for regulated desired changes in cholesterol metabolism

### EXAMPLE 4

### In Vivo Demonstration of the Cholesterol Lowering Properties of Cranberry and Grapefruit Derivatives

The following studies were performed to examine the cholesterol lowering properties of cranberry and grapefruit derivatives when administered to a mammal.

In particular, four different cranberry and grapefruit extracts (i.e., decharacterized cranberry, concentrated (2×) cranberry juice, processed grapefruit peel, and concentrated (2×) grapefruit juice) were prepared using methods described herein and tested for their cholesterol-lowering potential using a rabbit model of hypercholesterolemia. Of the cranberry and grapefruit extracts tested, at least one extract, processed grapefruit peel, produced desired effects on cholesterol metabolism when administered to a mammal.

The animal study was conducted as follows. First, rabbits where chosen as an model animal system because experimental hypercholesterolemia associated with an elevation of LDL cholesterol levels, similar to that observed in humans, can be induced by feeding the animals a low fat, cholesterol-free semipurified diet containing casein for at least 3 weeks. To establish whether this effect can be counteracted by any of the cranberry or grapefruit derivatives, the animals were given casein-based diets in which these products were incorporated. After 3 weeks, cholesterolemic responses were then measured in the test animals and compared to the control animals.

The particular animals used were New Zealand White male rabbits weighing 1.6-1.7 kg at the inception of the study and housed individually at constant temperature (21-24° C.) and under standard light cycle conditions (12h light: 12h dark). The animals were fed ground high fiber rabbit pellets for five days after arrival and then gradually transferred (over the course of one week) to the test diets. The animals were maintained on the diets for three weeks and food and fluid consumption as well as body weight changes were closely monitored.

Specifically, rabbits were assigned to five groups comprising eight animals each. The control group (CON), the group fed decharacterized cranberry (PRESS), and the group fed grapefruit pellets (PELL) was given semipurified, casein-based diets with or without respective supplements (30% each) and water to drink. The remaining two groups were given casein-based diets and either single strength cranberry juice (CRJUC) or double strength pink grapefruit juice (PGJUC) to drink. To ensure that the intake of sugars, fiber, and other components present in the administered cranberry and grapefruit derivatives did not reduce the intake of casein and other essential nutrients in the test animals, semipurified diets were modified to allow for the amounts of nutrients present in the cranberry and grapefruit derivatives. The composition of the semipurified diets alone (CON) or with cranberry or grapefruit derivative supplements is shown in Table 8. The results of nutrient analyses of the cranberry and grapefruit derivatives tested are presented in Table 9.

TABLE 8
Percent composition of semipurified diets used in the study.

| Ingredient | Control | PRESS | PELL | CRJUC | PGJUC |
|---|---|---|---|---|---|
| Casein | 26.30 | 37.60 | 37.60 | 31.60 | 30.70 |
| Dextrose | 48.20 | 32.70 | 45.10 | 37.90 | 39.60 |
| Alfacell | 13.00 | 11.95 | — | 15.60 | 15.20 |
| Salt mixture (P & H) | 4.00 | 5.70 | 5.70 | 4.80 | 4.67 |
| Molasses 1:1 | 3.00 | 4.30 | 4.30 | 3.60 | 3.48 |
| Vit. Mix (water sol.) | 1.5 | 2.15 | 2.15 | 1.80 | 1.75 |
| Vit. Mix (fat sol.) | 0.45 | 0.58 | 0.58 | 0.48 | 0.52 |
| Soy oil | 2.22 | 3.10 | 2.75 | 2.67 | 2.58 |
| Palm oil | 1.34 | 1.90 | 1.70 | 1.62 | 1.57 |
| Kcal/g | 3.26 | 3.22 | 3.66 | 3.12 | 3.14 |

PRESS-plus 30% ground decharacterized cranberry. With this supplement, the diet contained 2.28 cal/g
PELL-plus 30% ground grapefruit pellets. With this supplement, the diet contained 2.61 cal/g
CRJUC-plus cranberry juice (1× strength) instead of drinking water
PGJUC-plus pink grapefruit juice (2× normal strength) instead of drinking water

TABLE 9
Percent composition of cranberry and pink grapefruit products
incorporated into experimental diets.

| | CR. | PGR | CR JUICE | PCR JUICE |
|---|---|---|---|---|
| Ingredient | PRESSCAKE | PELLETS | (1x conc) | (2x conc) |
| Protein | 1.3 | 5.2 | — | — |
| Sugars | 0.4 | — | 2.73 | 16.4 |
| Fiber | 15.4 | 71.3 | — | — |
| Minerals | 0.1 | 10.2 | — | — |
| Fat | 0.2 | 1.5 | — | — |
| Moisture | 83.0 | 11.8 | — | — |
| Kcal/g | 0.085 | 0.34 | 0.11 | 0.65 |

The growth performance for all the animal groups tested are shown in Table 10. Food consumption was comparable to the control (CON) in test animals given decharacterized cranberry (PRESS), processed grapefruit peel (PELL), and cranberry juice (CRJUC) diet but lower in animals administered the pink grapefruit juice (PGJUC) diet. Intake of casein and fiber was generally similar among all the test animals due to modifications of diet composition. Total caloric intake was significantly increased in test animals fed decharacterized cranberry (PRESS) and processed grapefruit peel (PEL,L) and reduced in the animals administered pink grapefruit juice (PGJUC). This, however, did not affect overall body weight gains, which remained similar, except among those animals administered the diet including cranberry juice (CRJUC).

TABLE 10
Growth performance of rabbits fed experimental diets.

| | | | | | | | | Total |
|---|---|---|---|---|---|---|---|---|
| | | Initial | Weight | Food | Drink | Casein | Fiber | Caloric |
| | | Weight | Gain | Consum. | Consum. | Intake | Intake | Intake |
| Group | N | (kg) | (g/day) | (g/day) | (g/day) | (g/day) | (g/day) | (Kcal/day) |
| CON | 8 | 1.64 ± 0.03 | 26.2 ± 3.9 | 105.0 ± 6.4 | 211.9 ± 25.5 | 27.6 ± 1.7 | 13.7 ± 0.8 | 342 ± 21 |
| PRESS | 8 | 1.64 ± 0.04 | 27.9 ± 2.5 | 120.4 ± 6.2 | 396.6 ± 15.2* | 31.7 ± 1.6 | 15.7 ± 0.8 | 275 ± 14* |
| PELL | 8 | 1.63 ± 0.07 | 30.2 ± 3.0 | 108.2 ± 3.5 | 228.4 ± 28.1 | 28.5 ± 0.9 | 23.2 ± 0.8* | 282 ± 9* |
| CRJUC | 8 | 1.64 ± 0.03 | 9.8 ± 4.1* | 95.1 ± 3.9 | 78.8 ± 7.4* | 30.1 ± 1.2 | 14.8 ± 0.6 | 305 ± 12 |
| PGJUC | 8 | 1.64 ± 0.03 | 17.7 ± 2.8 | 68.7 ± 7.5* | 95.5 ± 6.0* | 21.1 ± 2.3* | 10.4 ± 1.1* | 278 ± 24* |

Means ± SEM. Statistical analysis by ANOVA plus post test by Dunnett's method.
*= significantly different from control, p <0.05.

Analysis for changes in cholesterol metabolism amongst all the animal groups was performed as follows. At the end of the study, the animals were fasted overnight. Next, blood samples were taken by heart puncture and fractions containing very low density lipoproteins (VLDL), low density lipoproteins (LDL), and high density lipoproteins (HDL) were separated from serum by discontinuous density gradient ultracentrifugation. Total cholesterol in each lipoprotein fraction and in whole serum was then measured using a standard enzymatic kit and values obtained for serum and lipoprotein cholesterol levels are presented in Table 11. The statistical analysis of the values presented was done by one-way ANOVA followed by Dunnett's post test and significant differences were reported at p<0.05 with outliers being removed from the data sets.

These experimental results indicate that the incorporation of cranberry (PRESS), cranberry juice (CRJUC), and pink grapefruit juice (PGJUC) into casein-based diet did not counteract the elevation of serum and undesirable LDL cholesterol produced by this diet. In contrast, incorporation of the processed grapefruit peel (PELL) significantly reduced serum total, reduced undesirable LDL cholesterol, as well as improving the LDL/HDL cholesterol ratio (i.e., a significant decrease). In addition, a significant reduction of undesirable LDL cholesterol levels, a significant increase of desirable HDL cholesterol levels, and an improvement (i.e., a significant decrease) in the LDL/HDL cholesterol ratio, were observed in the test animals administered decharacterized cranberry (PRESS). The test animals administer pink grapefruit juice (PGJUC) tended to have lower undesirable LDL cholesterol levels (i.e., a 26% reduction) than control animals (CON) as well as improved LDL/HDL cholesterol ratios as compared to controls. Cholesterolemic responses appeared to be largely unaffected in test animals administered cranberry juice (CRJUC).

TABLE 11
Serum and lipoprotein cholesterol levels in rabbits fed experimental diets.

| | | | VLDL | | | |
|---|---|---|---|---|---|---|
| | | Serum total | choles- | LDL | HDL | LDL/HDL |
| | | cholesterol | terol | cholesterol | cholesterol | cholesterol |
| Diet | N | mg/dL | mg/dL | mg/dL | mg/dL | ratio |
| CON | 6 | 146 ± 12 | 41 ± 5 | 80 ± 11 | 30 ± 3 | 2.8 ± 0.5 |
| PRESS | 7 | 116 ± 4 | 22 ± 2 | 48 ± 3* | 43 ± 1* | 1.1 ± 0.1* |
| PELL | 8 | 102 ± 11* | 36 ± 8 | 30 ± 3* | 33 ± 3 | 1.0 ± 0.2* |
| CRJUC | 7 | 169 ± 12 | 48 ± 5 | 87 ± 12 | 37 ± 4 | 2.5 ± 0.5 |
| PGJUC | 7 | 142 ± 10 | 42 ± 2 | 59 ± 4 | 37 ± 4 | 1.7 ± 0.2* |

Means ± SEM. Statistical analysis by ANOVA plus post test by Dunnett's method.
*= significantly different from control, p <0.05.

In summary, the above in vivo results demonstrated that administering processed grapefruit peel to an animal on a casein diet can counteract hypercholesterolemia induced by casein. This effect is unlikely due to merely an accompanying increase in fiber intake in these test animals because previous studies in rabbits have shown that dietary fiber does not affect casein-induced hypercholesterolemia.

Similarly, administering pink grapefruit juice into a casein diet also tended to counteract hypercholesterolemic responses induced by casein. Accordingly, it was concluded that certain grapefruit derivatives (and thus components thereof), when administered to a mammal, can improve the in vivo cholesterol profile of such a mammal.

### EXAMPLE 5

### Analysis of the Components of Decharacterized Cranberry

In this example, Tomah presscake (decharacterized cranberry) was determined to be enriched for a number of desirable therapeutic components. Accordingly, a detailed description of the major and minor components present in decharacterized cranberry, and suitable uses thereof, is described in the following subsections.

General Characterization of Major Components

In order to determine what major components of the decharacterized cranberry of the invention contributed to the beneficial anticancer effects when administered to a mammal (see, Example 2), a detailed analysis of the major components present in the decharacterized cranberry of the invention was performed. The decharacterized cranberry of the invention is preferably prepared using the methods described in, e.g., U.S. Pat. Nos. 5,320,861; 5,419,251. The amount of major components such as anthocyanins, phenolics, and proanthocyanidins of a decharacterized cranberry prepared using these methods (the so-called “Tomah presscake”) is surprisingly enriched over the levels of these compounds found in decharacterized cranberrys prepared by conventional methods (see Table 12).

TABLE 12
Comparison of Levels of Major Components in the Tomah Presscake as
Compared to Presscake Prepared by Conventional Method

| | Percent Available in Fruit | | |
|---|---|---|---|
| | Total | Total | Proantho- |
| | Anthocyanins | Phenolics | cyanidins |
| Sample | % | % | % |
| MARKHAM PRESSCAKE | 6.88% | 5.37% | 22.72% |
| TOMAH PRESSCAKE | 34.34% | 10.52% | 64.56% |

TABLE 13
Phenolic compounds identified in decharacterized cranberry.

| | Compound |
|---|---|
| 1 | Para-coumaric acid |
| 2 | Caffeic acid |
| 3 | Chlorogenic acid |
| 4 | Ferulic acid |
| 5 | Protocatechuic acid |
| 6 | Cinnamic acid |
| 7 | Benzoic acid |
| 8 | Gallic acid |
| 9 | Para-hydroxybenzoic acid |

The biologic properties of these compounds identified as being present in the cranberry are listed in a Table 15.

Analysis of the Flavanoid Compounds

The flavonoids in the decharacterized cranberry of the invention were determined using HPLC, MS, and UV spectral analysis. Standard HPLC parameters were employed and the chromnatogram was monitored at 280 or 320 nm.

Using the above methods of analysis, nine different phenolic components were identified and these are listed in Table 14 below.

TABLE 14
Flavonoids identified in decharacterized cranberry.

| | Class | Compound |
|---|---|---|
| 1 | Proanthocyanidins | polymers of flavan-3-ols |
| 2 | | epicatechin oligomers |
| 3 | | procyanidin B1 |
| 4 | | procyanidin b2 |
| 5 | | procyanidin b3 |
| 6 | Flavan-3-ols | catechin |
| 7 | | catechin gallate |
| 8 | | epicatechin |
| 9 | | epicatechin gallate |
| 10 | | epigallocatechin gallate |
| 11 | | gallocatechin gallate |
| 12 | Anthocyanins | cyanidin-3-arabinoside |
| 13 | | cyanidin-3-galactoside |
| 14 | | cyanidin-3-glucoside |
| 15 | | peonidin-3-arabinoside |
| 16 | | peonidin-3-galactoside |
| 17 | | peonidin-3-glucoside |
| 18 | | malvidin-3-arabinoside |
| 19 | | malvidin-3-glucoside |
| 20 | Flavonols | quercetin |
| 21 | | q-3-arabinoside (avicularin) |
| 22 | | q-3-galactoside (hyperin) |
| 23 | | q-3-glucoside (isoquercitrin) |
| 24 | | q-3-rhamnoside (quercitrin) |
| 25 | | myricetin |
| 26 | | m-3-arabinoside |
| 27 | | m-3-rhamnoside (myricitrin) |
| 28 | | m-3-digalactoside |
| 29 | | kaempferol |
| 30 | | isorhamnetin |

Quercetin in particular has been shown to inhibit tumor promotion by chemical carcinogens, inactivate some enzymes involved in the metabolism of carcinogens, and inhibit LDL oxidation.

The biologic properties of this and other related compounds identified as being present in the cranberry are listed in a Table 15.

Analysis of Fiber

The fiber, i.e., pectins in the decharacterized cranberry of the invention were also determined using standard techniques. These compounds have the beneficial properties of reducing colon cancer and plasma cholesterol concentration.

Analysis of Omega-3-fatty Acids and Tocotrienols

Decharacterized cranberry of the invention was also analyzed for the presence of omega-3-fatty acids and tocochromanols, a class of compounds that includes both tocopherols and tocotrienols. The method used for quantitating these compounds is based on the ability of these compounds to reduce the ferric ions (Fe³⁺) to (Fe²⁺). Absorption intensity is proportional to concentration, thus allowing for a determination of the amount of compound present in the sample.

Separation of individual tocotrienols was carried out using high performance liquid chromatography according to the method of Carpenter (Carpenter, Jr., A. P. J. Amer. Oil Chemists's Soc., 56:668 (1979)). Detecting particular tocotrienols was conducted using UV absorption at 295 mn and identification was achieved by performing a comparison of retention times for the unknown components against known standards.

Tocotrienols in particular can inhibit growth and proliferation in vitro of both estrogen receptor positive or negative human breast cancer cells. The biologic properties of these compounds identified as being present in decharacterized cranberry are listed in a Table 15.

Analysis of Triterpenoids

The decharacterized cranberry of the invention was also determined to contain triterpenoids, in particular, ursolic acid. The presence of these compounds was determined using art recognized techniques. Ursolic acid has been shown to have anti- inflammatory activity.

The biologic properties of these compounds identified as being present in the cranberry are listed in a Table 15.

Analysis of Other Components

The decharacterized cranberry of the invention was also determined to have several other compounds outside the compound classes mentioned above. For examples ellagic acid was also detected using are recognized techniques. This compound can stimulate apoptosis in cells and thus control the proliferation of cancer cells.

The biologic properties of these compounds identified as being present in the cranberry are listed in a Table 15.

Summary

The determinations described above reveal that the decharacterized cranberry of the invention has a remarkably high amount of anthocyanins, phenolics, and proanthocyanidins. In addition, decharacterized cranberry also contains a number of components having activity in a variety of pathways of cancer initiation, propagation, and proliferation. Accordingly, decharacterized cranberry can be considered a valuable source of therapeutic components and thus, a convenient vehicle for administering therapeutically effective mixtures of these components to a mammal, such as a human, having, e.g., cancer or hypercholesterolemia. A comprehensive list of the components present in decharacterized cranberry including therapeutic uses and applications for such components, is presented in Table 15.

TABLE 15

| COMPOUND | BIOLOGICAL ACTIVITY | UTILITY |
|---|---|---|
| CAFFEIC ACID | ABORTIFACIENT EFFECT | ANTIFERTILITY |
| CYANIDIN | ADENOSINE DEAMINASE INHIBITION | IMMUNODEFICIENCY |
| DELPHINIDIN | ANTIANAPHYLACTIC ACTIVITY | ALLERGY |
| HYPEROSIDE | ADENOSINE A-1 RECEPTOR BINDING | MUSCLE ACTIVITY |
| | INHIBITION | |
| KAEMPFEROL | 12-HETE SYNTHESIS INHIBITION | ANALGESIC ACTIVITY |
| KAEMPFEROL GLYCOSIDE A | ANTIIMPLANTATION EFFECT; | IMMUNOLOGIC; FERTILITY |
| | ANTISPERMATOGENIC EFFECT | |
| KAEMPFEROL GLYCOSIDE B | ANTIIMPLANTATION EFFECT | IMMUNOLOGIC |
| KAEMPFEROL TETRAACETATE | PLATELET AGGREGATION INHIBITION | PLATELET AGGREGATION |
| KAEMPFEROL TRIMETHYL ETHER | ANTIMUTAGENIC ACTIVITY | ANTIMUTAGENIC ACTIVITY |
| KAEMPFEROL, 3-4-7-TRIMETHYL | CYCLIC AMP INHIBITION | ANTIINFLAMMATORY |
| KAEMPFEROL, 3-4-7-TRI-O-METHYL | COMPLEMENT CLASSICAL PATHWAY | IMMUNITY |
| | INHIBITION; COMPLEMENT-ALTERNATE | |
| | PATHWAY STIMULATION | |
| KAEMPFEROL, 3-4′-DI-O-METHYL | COMPLEMENT CLASSICAL PATHWAY | IMMUNITY |
| | INHIBITION; COMPLEMENT-ALTERNATE | |
| | PATHWAY STIMULATION | |
| KAEMPFEROL, 3-O-METHYL | CYTOTOXIC ACTIVITY | ANTICANCER |
| KAEMPFEROL, 3-RUTINOSYL | RADICAL SCAVENGING EFFECT | ANTIOXIDANT |
| KAEMPFEROL, 4′-5-7-TRI-O- | ANTIBACTERIAL ACTIVITY; | ANTIBACTERIAL ACTIVITY; |
| METHYL: (FLAVONOID) | ANTIMYCOBACTERIAL ACTIVITY; | ANTIMYCOBACTERIAL |
| | PROSTAGLANDIN SYNTHESIS | ACTIVITY; |
| | INHIBITION | ANTIINFLAMMATORY |
| KAEMPFEROL, 6-8-DI-C-METHYL: 3- | PROSTAGLANDIN SYNTHESIS | ANTIINFLAMMATORY |
| METHYL ETHER (FLAVONOID) | INHIBITION | |
| KAEMFFEROL, 6-HYDROXY: | DNA POLYMERASE I INHIBITION; | ANTITUMOR; MUSCLE; |
| (FLAVONOID) | INOTROPIC EFFECT POSITIVE; REVERSE | ANTICANCER |
| | TRANSCRIPTASE INHIBITION | |
| KAEMPFEROL, 6-HYDROXY: 3-6-7- | INOTROPIC EFFECT POSITIVE | MUSCLE |
| TRIGLUCOSIDE (FLAVONOID) | | |
| KAEMPFEROL, 6-HYDROXY: 3-6-DI- | INOTROPIC EFFECT POSITIVE | MUSCLE |
| GLUCOSIDE (FLAVONOID) | | |
| KAEMPFEROL, 6-HYDROXY: 3-O- | ATP-ASE(Na+/k+) INHIBITION; | BLOOD; MUSCLE |
| BETA-C)-GLUCOSIDE (FLAVONOID) | INOTROPIC EFFECT POSITIVE | |
| KAEMPFEROL, 6-HYDROXY: 3- | INOTROPIC EFFECT POSITIVE | MUSCLE |
| RUTINOSYL-6-GLUCOSIDE | | |
| (FLAVONOID) | | |
| KAEMPFEROL, 6-HYDROXY: 4- | CYTOTOXIC ACTIVITY | ANTICANCER |
| METHYL ETHER 3-7- | | |
| DIHRHAMNOSIDE (FLAVONOID) | | |
| KAEMPFEROL, 6-HYDROXY-3-7- | SMOOTH MUSCLE RELAXANT ACTIVITY; | MUSCLE; NERVOUS SYSTEM |
| DIMETHYL ETHER (FLAVONOID) | SPASMOLYTIC ACTIVITY | |
| KAEMPFEROL, 6-METHOXY: | ANTICYTOTOXIC ACTIVITY; ATP-ASE | ANTICANCER; BLOOD; |
| (FLAVONOID) | (Ca++) INHIBITION; BETA- | DETOXIFICATION; |
| | GLUCURONIDASE INHIBITION; | ANTICANCER; ANTIOXIDANT |
| | CYTOTOXIC ACTIVITY; HISTAMINE | |
| | RELEASE INHIBITION | |
| KAEMPFEROL, 6-METHOXY: 3-7- | ANTIVIRAL ACTIVITY | ANTIVIRAL ACTIVITY |
| DIMETHYL ETHER (FLAVONOID) | | |
| KAEMPFEROL, 6-METHOXY: 3- | ANTIVIRAL ACTIVITY | ANTIVIRAL ACTIVITY |
| METHYL ETHER (FLAVONOID) | | |
| KAEMPFEROL, 6-METHOXY: 3-O- | CALCIUM ION RELEASE INHIBITION; | BLOOD; ANTICANCER |
| BETA-G-(1-6)-ROBINOBIOSIDE | CYTOTOXIC ACTIVITY | |
| (FLAVONOID) | | |
| KAEMPFEROL, 6-METHOXY: 3-O- | ALDOL REDUCTASE (LENS) INHIBITION | ANTINEOPLASTIC |
| BETA-D-ROBINOBIOSIDE | | |
| (FLAVONOID) | | |
| KAEMPFEROL-3-(2-3-DIACETOXY-4- | ANTIFUNGAL ACTIVITY | ANTIFUNGAL ACTIVITY |
| PARA-COUMAROYL)-RHOMNOSIDE | | |
| (FLAVONOID) | | |
| KAEMPFEROL-3-4′-7-TRIMETHYL | ANTICRUSTACEAN ACTIVITY; ANTIVIRAL | ANTICRUSTACEAN ACTIVITY; |
| ETHER (FLAVONOID) | ACTIVITY; CYTOTOXIC ACTIVITY | ANTIVIRAL ACTIVITY; |
| | | ANTICANCER |
| KAEMPFEROL-3-4′-DIMETHYL | ANTIVIRAL ACTIVITY; REVERSE | ANTIVIRAL ACTIVITY; |
| ETHER (FLAVONOID) | TRANSCRIPTASE INHIBITION | ANTICANCER |
| KAEMPFEROL-3-7-DIMETHYL | ANTIVIRAL ACTIVITY | ANTIVIRAL ACTIVITY |
| ETHER (FLAVONOID) | | |
| KAEMPFEROL-3-7-DI-O-BETA-D- | CITRATE SYNTHASE INHIBITION | ANTIOXIDANT |
| GLUCOSIDE (FLAVONOID) | | |
| KAEMPFEROL-3-BETA- | PLANT ROOT GROWTH INHIBITION | PLANT GROWTH |
| DIRHAMNOSIDE (FLAVONOID) | | |
| KAEMPFEROL-3-GLUCOSIDE-7- | ANTIOXIDANT ACTIVITY; | ANTIOXIDANT; |
| RHAMNOSIDE (FLAVONOID) | CYCLOOXYGENASE STIMULATION; | ANTIINFLAMMATORY; |
| | LIPOXYGENASE INHIBITION; PLATELET | ANTIOXIDANT; PLATELET |
| | AGGREGATION INHIBITION | AGGREGATION |
| KAEMPFEROL-3- | BRONCHODILATOR ACTIVITY | LUNG |
| NEOHESPERIDOSIDE (FLAVONOID) | | |
| KAEMPFEROL-3-O-(2-4-(DI-PARA- | ANTIINFLAMMATORY ACTIVITY | ANTIINFLAMMATORY ACTIVITY |
| COUMAROYL)-BETA-D-GLUCOSIDE) | | |
| (FLAVONOID) | | |
| KAEMPFEROL-3-O-(2″-6″-DI-O- | REPELLENT ACTIVITY (MOLLUSK) | MOLLUSK |
| TRANS-PARA-COUMAROYL-BETA- | | |
| D-GLUCOSIDE) (FLAVONOID) | | |
| KAEMPFEROL-3-O-(2″-O-GALLOYL)- | ANGIOTENSIN-CONVERTING ENZYME | VASOPRESSOR |
| GLUCOSIDE (FLAVONOID) | INHIBITION | |
| KAEMPFEROL-3-O-(6″-ACETYL)- | CHROMOSOME ABERRATION INHIBITION | ANTICANCER |
| BETA-D-GLUCOSIDE (FLAVONOID) | | |
| KAEMPFEROL-3-O-(6″-O-ACETYL)- | GLUCOSYLTRANSFERASE INHIBITION | LIVER |
| BETA-D-GLUCOSIDE (FLAVONOID) | | |
| KAEMPFEROL-3-O-(6″-O-ACETYL)- | CYTOTOXIC ACTIVITY | ANTICANCER |
| GLUCOSIDE (FLAVONOID) | | |
| KAEMPFEROL-3-O-(6″-O-TRANS- | ANTIVIRAL ACTIVITY; CYTOTOXIC | ANTIVIRAL ACTIVITY; |
| PARA-COUMAROYL)-BETA-D- | ACTIVITY | ANTICANCER |
| GLUCOPYRANOSIDE (FLAVONOID)- | | |
| KAEMPFEROL-3-O-[2-O-ALPHA-L- | PLATELET AGGREGATION INHIBITION | PLATELET AGGREGATION |
| RHAMNOPYRANOSYL-4-O-BETA-D- | | |
| GLUCOPYRANOSYL]-BETA | | |
| KAEMPFEROL-3-O-ALPHA-(6″- | FEEDING DETERRENT (INSECT) | INSECTICIDAL |
| PARAPCOUMAROYL-GLUCOSYL- | | |
| BETA-1-2-RHAMNOSIDE) | | |
| (FLAVONOID) | | |
| KAEMPFEROL-3-O-ALPHA- | PROTEIN TYROSINE KINASE INHIBITION | VASCULAR |
| ARABIINOPYRANOSIDE | | |
| (FLAVONOID) | | |
| KAEMPFEROL-3-O-ALPHA-L-(2″-3″- | ANTIBACTERIAL ACTIVITY; | ANTIBACTERIAL ACTIVITY; |
| DI-TRANS-PARA-COUMAROYL)- | ANTIBACTERIAL ACTIVITY; CYTOTOXIC | ANTIBACTERIAL ACTIVITY; |
| RHAMNOPYRANOSIDE | ACTIVITY | ANTICANCER |
| (FLAVONOID) | | |
| KAEMPFEROL-3-O-ALPHA-L- | ANTIBACTERIAL ACTIVITY; ANTIVEAST | ANTIBACTERIAL ACTIVITY; |
| ARABINOFURANOSYL-7-O-ALPHA- | ACTIVITY | ANTIYEAST |
| L-RHAMNOPYRANOSIDE | | |
| (FLAVONOID) | | |
| KAEMPFEROL-3-O-ALPHA-L- | ANALGESIC ACTIVITY | ANALGESIC ACTIVITY |
| RHAMNOPYRANOSYL-(1-6)-BETA-D- | | |
| GLUCOPYRANOSIDE (FLAVONOID) | | |
| KAEMPFEROL-3-O- | ANTIOXIDANT ACTIVITY | ANTIOXIDANT |
| ARABINOPYRANOSIDE-2″-GALLATE | | |
| (FLAVONOID) | | |
| KAEMPFEROL-3-O-BETA-D- | ANTIBACTERIAL ACTIVITY | ANTIBACTERIAL ACTIVITY |
| GLUOCOPYRANOSYL(1-2)-(ALPHA- | | |
| L-RHAMNOPYRANSOYL(1-6))-BETA | | |
| KAEMPFEROL-3-O-BETA-D- | VASODILATOR ACTIVITY | VASCULAR |
| GLUCOSIDE-7-RHAMNOSIDE | | |
| (FLAVONOID) | | |
| KAEMPFEROL-3-O-BETA-D- | PLATELET AGGREGATION INHIBITION | PLATELET AGGREGATION |
| NEOHESPERIDOSIDE (FLAVONOID) | | |
| KAEMPFEROL-3-O-BETA-D- | XANTHINE OXIDASE INHIBITION | ANTIOXIDANT |
| RUTINOSIDE (FLAVONOID) | | |
| KAEMPFEROL-3-O-BETA-D- | ANALGESIC ACTIVITY; | ANALGESIC ACTIVITY; |
| SOPHOROSIDE (FLAVONOID) | ANTIINFLAMMATORY ACTIVITY; | ANTIINFLAMMATORY |
| | BRONCHODILATOR ACTIVITY | ACTIVITY; LUNG |
| KAEMPFEROL-3-O-BETA-D- | LIPID PEROXIDE FORMATION INHIBITION | ANTIOXIDANT |
| XYLOFURANOSIDE (FLAVONOID) | | |
| KAEMPFEROL-3-O-BETA-D- | LIPID PEROXIDE FORMATION INHIBITION | ANTIOXIDANT |
| XYLOFURANOSYL(1-2)- | | |
| GALACTOPYRANOSIDE | | |
| (FLAVONOID) | | |
| KAEMPFEROL-3-O-BETA-D- | EXOCYTOSIS STIMULATION | ANTICANCER |
| XYLOFURANOSYL(1-2)-BETA-D- | | |
| ARABINOPYRANOSIDE | | |
| (FLAVONOID) | | |
| KAEMPFEROL-3-O-METHYL ETHER | XANTHINE OXIDASE INHIBITION | ANTIOXIDANT |
| (FLAVONOID) | | |
| KAEMPFEROL-3-O- | ANTIBACTERIAL ACTIVITY; | ANTIBACTERIAL ACTIVITY; |
| NEOHESPERIDOSIDE (FLAVONOID) | ANTIOXIDANT ACTIVITY; ANTIYEAST | ANTIOXIDANT; ANTIVEAST |
| | ACTIVITY | |
| KAEMPFEROL-3-O-RHAMNOSIDE | COMPLEMENT ALTERNATEIVE | IMMUNITY; ANTIOXIDANT |
| (FLAVONOID) | PATHWAY INHIBITION; COMPLEMENT | |
| | CLASSICAL PATHWAY INHIBITION; | |
| | COMPLEMENT ALTERNATE PATHWAY | |
| | STIMULATION; LIPID PEROXIDE | |
| | FORMATION INHIBITION | |
| KAEMPFEROL-3-O-RHAMNOSYL- | ANTIPRURITIC ACTIVITY | ALLERGY |
| DIGLUCOSIDE (FLAVONOID) | | |
| KAEMPFEROL-3-O-RUTINOSIDE | ADENOSINE A-1 RECEPTOR BINDING | MUSCLE ACTIVITY; |
| (FLAVONOID) | INHIBITION; ANTIBACTERIAL ACTIVITY; | ANTIBACTERIAL ACTIVITY; |
| | ANTIYEAST ACTIVITY; CHRONOTROPIC | ANTIYEAST; HEART; |
| | EFFECT NEGATIVE; COMPLEMENT | IMMUNITY; IMMUNITY; |
| | ALTERNATIVE PATHWAY INHIBITION; | CANCER; HYPERTENSION; |
| | COMPLEMENT-ALTERNATE PATHWAY | MUSCLE; NERVOUS SYSTEM |
| | STIMULATION; EPSTEIN-BARR VIRUS | |
| | EARLY ANTIGEN ACTIVATION | |
| | INHIBITION; HYPOTENSIVE ACTIVITY; | |
| | SMOOTH MUSCLE RELAXANT ACTIVITY; | |
| | SPASMOLYTIC ATIVITY | |
| KAEMPFEROL-3-O-SOPHOROSIDE | LIPID PEROXIDE FORMATION INHIBITION | ANTIOXIDANT |
| (FLAVONOID) | | |
| KAEMPFEROL-3-RHAMNOSIDE | ANTIOXIDANT ACTIVITY; | ANTIOXIDANT; |
| (FLAVONOID) | CYCLOOXYGENASE STIMULATION; | ANTIINFLAMMATORY; |
| | LIPOXYGENASE INHIBITION | ANTIOXIDANT |
| KAEMPFEROL-3-O-RHAMNOSYL-7- | ANTIMUTAGENIC ACTIVITY | ANTIMUTAGENIC ACTIVITY |
| GLUOSIDE (FLAVONOID) | | |
| KAEMPFEROL-3-RUTINOSIDE | ANTICYTOTOXIC ACTIVITY; AUTOPHAGY | ANTICANCER; IMMUNOLOGY; |
| (FLAVONOID) | STIMULATION; CYTOTOXIC ACTMTY; | ANTICANCER; MUSCLE |
| | INOTROPIC EFFECT POSITIVE | |
| KAEMPFEROL-3-SULFATE | GLUCOSE TRANSPORT STIMULATION | DIABETES |
| (FLAVONOID) | | |
| KAEMPFEROL-4′-O-BETA-D- | HYPERTENSIVE ACTIVITY | HYPERTENSION |
| GLUCOSIDE (FLAVONOID) | | |
| KAEMPFEROL-4-O-METHYL ETHER | ANTICHOLINERGIC ACTIVITY; | NERVOUS SYSTEM; |
| (FLAVC)NOID) | ANTIINFLAMMATORY ACTIVITY; | ANTIINFLAMMATORY |
| | BARBITURATE POTENTIATION; | ACTIVITY; DRUG; HEART |
| | CHRONOTROPIC EFFECT POSITIVE | |
| MALVIDIN | ABORTIFACIENT EFFECT | ANTIFERTILITY |
| MYRICIETIN | 12-HETE SYNTHESIS INHIBITION | ANALGESIC ACTIVITY |
| MYRICETIN | PLANT ROOT GROWTH INHIBITION | PLANT GROWTH |
| MYRICENTIN RHAMNOSIDE | ANTIULCER ACTIVITY; SMOOTH MUSCLE | ANTIULCER; MUSCLE |
| | RELAXANT ACTIVITY | |
| MYRICETIN TRIMETHYL ETHER | 12-HETE SYNTHESIS INHIBITION; | ANALGESIC ACTIVITY; |
| | SPASMOLYTIC ACTIVITY | NERVOUS SYSTEM |
| MYRICETIN-3-ARABINOSIDE | ANTIOXIDNT ACTMTY; | ANTIOXIDANT; |
| | CYCLOOXYGENASE STIMULATION; | ANTIINFLAMMATORY; |
| | LIPOSYGENASE INHIBITION | ANTIOXIDANT |
| MYRICETIN-3-O-ALPHA-L- | XANTHINE OXIDASE INHIBITION | ANTIOXIDANT |
| MYRICETIN-3-O-ARABINOSIDE | CYTOTOXIC ACTIVITY; HISTAMINE | ANTICANCER; ANTIOXIDANT |
| | RELEASE INHIBITION | |
| MYRICETIN-3-O-BETA-D- | ANTIOXIDANT ACTIVITY; ANTIOXIDANT; | |
| | CYCLOOXYGENASE STIMULATION; | ANTIINFLAMMATORY; |
| | LIPOXYGENASE INHIBITION | ANTIOXIDANT |
| MYRICETIN-3-O-BETA-D- | ANTIINFLAMMATORY ACTIVITY; | ANTIINFLAMMATORY |
| GLUCURONIDE | CYCLOOXYGENASE 1 INHIBITION; | ACTIVITY; |
| | CYCLOOXYGENASE 2 INHIBITION; | ANTIINFLAMMATORY; |
| | LIPOXYGENASE,5: INHIBITION; | ANTIINFLAMMATORY; |
| | LIPOXYGENASE,5: INHIBITION; | ANTIOXIDANT; ANTIOXIDANT; |
| | PLATELET AGGREGATION INHIBITION; | PLATELET AGGREGATION; |
| | PROSTAGLANDIN SYNTHESIS | ANTIINFLAMMATORY; |
| | INHIBITION; ULCEROGENIC ACTIVITY | DIGESTION |
| MYRICETIN-3-O-BETA-D-SYLOSIDE | CYTOTOXIC ACTIVITY; HISTAMINE | ANTICANCER; ANTIOXIDANT; |
| | RELEASE INHIBITION | ANTIOXIDANT |
| OLEANOLIC ACID | ACID PHOSPHATASE INHIBITION; | ANTINEOPLASTIC; |
| | ADENOSINE DEAMINASE INHIBITION; | IMMUNODEFICIENCY; BONE |
| | ADRENAL HYPERTROPHY EFFECT | DISEASE |
| PELARGONIDIN | ANTIANAPHYLACTIC ACTIVITY; | ALLERGY; ANTIMUTAGENIC |
| | ANTIMUTAGENIC ACTIVITY ACTIVITY | |
| PELARGONIDIN-3-O-BETA-D- | ANTIANAPHYLACTIC ACTIVITY; | ALLERGY; ANTIOXIDANT; |
| GLUCOSIDE | ANTIOXIDANT ACTIVITY; HYDROXIDE | ANTIOXIDANT; ANTIOXIDANT; |
| | RADICAL GENERATION INHIBITION; | ANTIOXIDANT |
| | LIPID PEROXIDE FORMATION | |
| | INHIBITION; RADICAL SCAVENGING | |
| | EFFECT | |
| PELARGONIDIN-5-7-DIMETHYL | ANTIHYPERGLYCEMIC ACTIVITY | DIABETES |
| ETHER 3-O-ALPHA-L-RHAMNOSIDE | | |
| PROANTHOCYANIDIN | ANTIFUNGAL ACTIVITY; CYTOTOXIC | ANTIFUNGAL ACTIVITY; |
| | ACTIVITY | ANTICANCER |
| PROCYANIDIN | ANTIFUNGAL ACTIVITY | ANTIFUNGAL ACTIVITY |
| QUERCETIN | 12-HETE SYNTHESIS INHIBITION | ANALGESIC ACTIVITY |
| QUERCETIN,4-7-DIMETHYL | XANTHINE OXIDASE INHIBITION | ANTIOXIDANT |
| QUERCETIN,4-7-DI-O-BENZOYL | ANTIVIRAL ACTIVITY (PLANT | ANTIVIRAL ACTIVITY |
| | PATHOGENS) | |
| QUERCETIN,4-7-DI-O-METHYL | MUTAGENIC ACTIVITY | ANTICANCER |
| QUERCETIN,5-7-DI-O-METHYL | MUTAGENIC ACTIVITY | ANTICANCER |
| QUERCETIN,5-O-METHYL | MUTAGENIC ACTIVITY; VIRAL | 7 ANTICANCER; INFECTION |
| | INFECTIVITY INHIBITION | |
| QUERCETIN,6-8-DI-C-METI-IYL: 3-7- | PROSTAGLANDIN SYNTHESIS | ANTIINFLAMMATORY |
| DIMETHYL ETHER | INHIBITION | |
| QUERCETIN,6-8-DI-C-METHYL: 3- | ANTIBACTERIAL ACTIVITY; | ANTIBACTERIAL ACTIVITY; |
| METHYL ETHER | PROSTAGLANDIN SYNTHESIS | ANTIINFLAMMATORY |
| | INHIBITION | |
| QUERCETIN,6-C-METHYL: 3-3′-7- | PROSTAGLANDIN SYNTHESIS | ANTIINFLAMMATORY |
| TRIMETHYL ETHER | INHIBITION | |
| QUERCETIN,6-C-METHYL: 3-7- | ANTIBACTERIAL ACTIVITY; | ANTIBACTERIAL ACTIVITY; |
| DIMETHYL ETHER | PROSTAGLANDIN SYNTHESIS | ANTIINFLAMMATORY |
| | INHIBITION | |
| QUERCETIN,6-C-METHYL: 3-METHYL | ANTIBACTERIAL ACTIVITY; | ANTIBACTERIAL ACTIVITY; |
| ETHER | PROSTAGLANDIN SYNTHESIS | ANTIINFLAMMATORY |
| | INHIBITION | |
| QUERCETIN,7-METHOXY: | ANTIMUTAGENIC ACTIVITY | ANTIMUTAGENIC ACTIVITY |
| QUERCETIN,7-O-BENZOYL: | GYCLIC GMP INHIBITION; | ANTIINFLAMMATORY; |
| | IMMUNOPRECIPITATION REACTION | IMMUNOLOGY |
| QUERCETIN, DIHYDRO: (DL) | ADENOSINE RECEPTOR BINDING | T MUSCLE ACTIVITY |
| | INHIBITION | |
| QUERCETIN, DIHYDRO: 3-O- | SWEETENING EFFECT | TASTE |
| ACETATE (FLAVONOID) | | |
| QUERCETIN, ISO: | REVERSE TRANSCRIPTASE INHIBITION; | ANTICANCER; NERVOUS |
| | SPASMOLYTIC ACTIVITY | SYSTEM |
| QUERCETIN,METHYL: (FLAVONOID) | RNA SYNTHESIS INHIBITION | ANTICANCER |
| QUERCETIN, PENTAACETYL: | PHOSPHODIESTERASE INHIBITION | ANTIINFLAMMATORY |
| (FLAVONOID) | | |
| QUERCETIN, PENTAHYDROXY- | CELL MEMBRANE PERMEATION | ANTICANCER |
| ETHYL: (FLAVONOID) | | |
| QUERCETIN, PENTAMETHOXY: | ANTIMUTAGENIC ACTIVITY | ANTIMUTAGENIC ACTIVITY |
| (FLAVONOID) | | |
| QUERCETIN, PENTAMETHYL: | ANTIVIRAL ACTIVITY; MUTAGENIC | ANTIVIRAL ACTIVITY; |
| (FLAVONOID) | ACTIVITY; PHOSPHODIESTERASE | ANTICANCER; |
| | INHIBITION; SPASMOLYTIC ACTIVITY; | ANTIINFLAMMATORY; |
| | SPASMOLYTIC ACTIVITY | NERVOUS SYSTEM; NERVOUS |
| | | SYSTEM |
| QUERCETIN, PENTA-O-ETHYL: | ANTICHOLINERGIC ACTIVITY; | NERVOUS SYSTEM; |
| (FLAVONOID) | ANTI HYPERTENSIVE ACTIVITY; | HYPERTENSION; HEART; |
| | CHRONOTROPIC EFFECT NEGATIVE; | HYPERTENSION; |
| | HYPERTENSIVE ACTIVITY; | HYPERTENSION |
| | HYPERTENSIVE ACTIVITY | |
| QUERCETIN, TETRAHYDROXY- | CELL MEMBRANE STABILIZER | ANTICANCER |
| ETHYL: (FLAVONOID) | | |
| QUERCETIN, TETRA-METHOXY: | ANTICLASTOGENIC ACTIVITY | CHROMOSOME ABNORMALITY |
| (FLAVONOID) | | |
| QUERCETIN-2-SULFATE | ALDEHYDE REDUCTASE INHIBITION; | ANTIOXIDANT; |
| (FLAVONOID) | ALDOL REDUCTASE (LENS) INHIBITION; | ANTINEOPLASTIC; |
| | NADH OXIDASE INHIBITION | ANTICANCER |
| QUERCETIN-3-(2″-GALLOYL- | MOLLUSCICIDAL ACTIVITY | MOLLUSCICIDAL ACTIVITY |
| GLUCOSIDE) (FLAVONOID) | | |
| QUERCETIN-3-(2″-O-GALLOYL)- | ANGIOTENSIN-CONVERTING ENZYME | VASOPRESSOR |
| GLUCOSIDE (FLAVONOID) | INHIBITION | |
| QUERCETIN-3-3-4-TRIMETHYL | AFLATOXIN INACTIVATION; | DNA BINDING; ANTITUMOR |
| EHTER (FLAVONOID) | GENOTOXICITY INHIBITION | |
| QUERCETIN-3-3′-7-TRIMETHYL | HISTAMINE RELEASE INHIBITION | ANTIOXIDANT |
| ETHER (FLAVONOID) | | |
| QUERCETIN-3-3′-DIMETHYL ETHER | ANTIANAPHYLACTIC ACTIVITY; | ALLERGY; ANTIOXIDANT |
| (FLAVONOIC) | HISTAMINE RELEASE INHIBITION | |
| QUERCETIN-3-4′-7-TRIMETHYL | ANTIVIRAL ACTIVITY (PLANT | ANTIVIRAL ACTIVITY |
| ETHER (FLAVONOID) | PATHOGENS) | |
| QUERCETIN-3-4′-DIGLUCOSIDE | ANTIOXIDANT ACTIVITY; LIPID | ANTIOXIDANT; ANTIOXIDANT |
| (FLAVONOID) | PEROXIDE FORMATION INHIBITION | |
| QUERCETIN-3-4′-DIMETHYL ETHER | SPASMOLYTIC ACTIVITY | NERVOUS SYSTEM |
| (FLAVONOID) | | |
| QUERCETIN-3-4-DI-O-BETA-D- | CATALASE INHIBITION; CELL | ANTIOXIDANT; ANTICANCER; |
| GLUCOSIDE | PROLIFERATION INHIBITION; | HEPATOPROTECTION; |
| | GLUTAMATE-OXALOAECETATE- | HEPATOPROTECTION; |
| | TRANSAMINASE INHIBITION; | IMMUNOLOGY; ANTICANCER; |
| | GLUTAMATE-OXALOACETATE- | ANTICANCER |
| | TRANSAMINASE STIMULATION; | |
| | IMMUNOSTIMULANT ACTIVITY; NADPH- | |
| | CYTOCHROME C REDUCTASE | |
| | STIMULATION; NADPH-CYTOCHROME C | |
| | REDUCTASE STIMULATION | |
| QUERCETIN-3-7-DIGLUCOSIDE | ANTIOXIDANT ACTIVITY; ATP-ASE | ANTIOXIDANT; BLOOD; |
| (FLAVC)NOID) | (NA+/K+) INHIBITION; | ANTIINFLAMMATORY; |
| | CYCLOOXYGENASE STIMULATION; | MUSCLE; ANTIOXIDANT |
| | INOTROPIC EFFECT POSITIVE; | |
| | LIPOXYGENASE INHIBITION | |
| QUERCETIN-3-ACETATE, DIHYDRO: | MUTAGENIC ACTIVITY; SWEETENING | ANTICANCER; TASTE |
| (FLAVONOID) | EFFECT | |
| QUERCETIN-3-ALPHA- | ANTIBACTERIAL ACTIVITY | ANTIBACTERIAL ACTIVITY |
| ARABINOPYRANOSIDE-2″-GALLATE | | |
| (FLAVONOID) | | |
| QUERCETIN-3-GENTIOBIOOSIDE | ANTIOXIDANT ACTIVITY | ANTIOXIDANT |
| (FLAVONOID) | | |
| QUERCETIN-3-GLUCOSIDE-7- | ANTIOXIDANT ACTIVITY; ANTIOXIDANT; | |
| RHAMNOSIDE | CYCLOOXYGENASE STIMULATION; | ANTIINFLAMMATORY; |
| | LIPOXYGENASE INHIBITION | ANTIOXIDANT |
| QUERCETIN-3-METHYL ETHER | ALDOL REDUCTASE (LENS) INHIBITION; | ANTINEOPLASTIC; ALLERGY; |
| (FLAVONOID) | ANTIANAPHYLACTIC ACTIVITY; | ANTIBACTERIAL ACTIVITY; |
| | ANTIBACTERIAL ACTIVITY; ANTIFUNGAL | ANTIFUNGAL ACTIVITY; |
| | ACTIVITY; ANTIOXIDANT ACTIVITY; | ANTIOXIDANT; ANTIVIRAL |
| | ANTIVIRAL ACTIVITY; HISTAMINE | ACTIVITY; ANTIOXIDANT; |
| | RELEASE INHIBITION; SUPEROXIDE | ANTIOXIDANT |
| | RADICAL SCAVENGING ACTIVITY | |
| QUERCETIN-3-O-(2″-6″-ALPHA-L- | OVIPOSITION STIMULATION | FERTILITY |
| DIRHAMNOPYRONOSYL)-BETA-D- | | |
| GALACTOPYRANOSIDE | | |
| (FLAVONOID) | | |
| QUERCETIN-3-O-(2″-6″-O- | HIV-1 INTEGRASE INHIBITION | AIDS |
| DIGALLOYL)-BETA-D- | | |
| GALACTOPYRANOSIDE | | |
| (FLAVONOID) | | |
| QUERCETIN-3-O-(2-BETA-D- | OVIPOSITION INHIBITION | FERTILITY |
| XYLOPRANOSYL-RUTINOSIDE) | | |
| (FLAVONOID) | | |
| QUERCETIN-3-O-(2′-GALLOYL)- | HIV-1 INTEGRASE INHIBITION | AIDS |
| ALPHA-L-ARABINOPYRANOSIDE | | |
| (FLAVONOID) | | |
| QUERCETIN-3-O-(2″-GALLOYL)- | HIV-1 INTEGRASE INHIBITION | AIDS |
| BETA-D-GALACTOPYRANOSIDE | | |
| QUERCETIN-3-O-(2″-GALLOYL)- | HEMOLYSIS INHIBITORY ACTIVITY | BLOOD |
| BETA-D-GLUCOSIDE (FLAVONOID) | | |
| QUERCETIN-3-O-(2″-O-BALLOYL)- | CYTOTOXIC ACTIVITY | ANTICANCER |
| BETA-D-GALACTOSIDE | | |
| (FLAVONOID) | | |
| QUERCETIN-3-O-(2′-O-GALLOYL- | ANGIOTENSIN-CONVERTING ENZYME | VASOPRESSOR; |
| GLUCOSIDE) (FLAVONOID) | INHIBITION; MOLLUSCICIDAL ACTIVITY | MOLLUSCICIDAL ACTIVITY |
| QUERCETIN-3-O-(6″-GALLOYL)- | HEMOLYSIS INHIBITORY ACTIVITY | BLOOD |
| BETA-D-GLUCOSIDE (FLAVONOID) | | |
| QUERCETIN-3-O-(6″-GALLOYL)- | SEROTONIN ANTAGONIST ACTIVITY; | BRAIN; NERVOUS SYSTEM |
| GLUCOSIDE (FLAVONOID) | SPASMOGENIC ACTIVITY | |
| QUERCETIN-3-O-(6″-O-ACETYL)- | GLUCOSYLTRANSFERASE INHIBITION; | LIVER; PLATELET |
| BETA-D-GLUCOSIDE (FLAVONOID) | PLATELET AGGREGATION INHIBITION | AGGREGATION |
| QUERCETIN-3-O-ALPHA-(6′-PARA- | FEEDING DETERRENT (INSECT) | INSECTICIDAL |
| COUMAROYL-GLUCOSYL-BETA-1-2- | | |
| RHAMNOSIDE) (FLAVONOID) | | |
| QUERCETIN-3-O-ALPHA-L- | REVERSE TRANSCRIPTASE INHIBITION | ANTICANCER |
| ARABINOPYRANOSIDE | | |
| (FLAVONOID) | | |
| QUERCETIN-3-O-ALPHA-I- | ANTIALLERGENIC ACTIVITY | ALLERGY |
| ARABINOPYRANOSYL(1-2)-ALPHA- | | |
| L-RHAMNOPYRANOSIDE | | |
| (FLAVONOID) | | |
| QUERCETIN-3-O- | CYTOTOXIC ACTIVITY; HISTAMINE | ANTICANCER; ANTIOXIDANT |
| ARABINOFURANOSIDE | RELEASE INHIBITION | |
| (FLAVONOID) | | |
| QUERCETIN-3-O-BETA-D- | ANTIBACTERIAL ACTIVITY; | ANTIBACTERIAL ACTIVITY; |
| FALACTOSE(1-6)-O-ALPHA-L- | TOPOISOMERASE II INHIBITION | ANTICANCER |
| RHAMNOSE (FLAVONOID) | | |
| QUERCETIN-3-O-BETA-D- | PLATELET AGGREGATION INHIBITION | PLATELET AGGREGATION |
| GALACTOSIDE-2″-GALLATE | | |
| (FLAVONOID) | | |
| QUERCETIN-3-O-BETA-D- | INTESTINAL MOTILITY INHIBITION | DIGESTION |
| GALACTURONIDE (FLAVONOID) | | |
| QUERCETIN-3-O-BETA-D- | MOLLUSCICIDAL ACTIVITY | MOLLUSCICIDAL ACTIVITY |
| GLUCOPYRANOSIDE-2″-GALLATE | | |
| (FLAVONOID) | | |
| QUERCETIN-3-O-BETA-D- | ANTIBACTERIAL ACTIVITY | ANTIBACTERIAL ACTIVITY |
| GLUCOPYRANOSYL(1-2)-BETA-D- | | |
| GLUCOPYRANOSIDE (FLAVONOID) | | |
| QUERCETIN-3-O-BETA-D- | OVIPOSITION STIMULATION | FERTILITY |
| GLUCOPYRANOSYL(1-6)-BETA-D- | | |
| GALACTOPYRANOSIDE | | |
| (FLAVONOID) | | |
| QUERCETIN-3-O-BETA-D- | ANGIOTENSIN-CONVERTING ENZYME | VASOPRESSOR; DIGESTION |
| GLUCORONIDE (FLAVONOID) | INHIBITION; INTESTINAL MOTILITY | |
| | INHIBITION | |
| QUERCETIN-3′-O-BETA-D- | ANTIBACTERIAL ACTIVITY; ANTIULCER | ANTIBACTERIAL ACTIVITY; |
| GLUCOSIDE | ACTIVITY; CITRATE SYNTHASE | ANTIULCER; ANTIOXIDANT; |
| | INHIBITION; SMOOTH MUSCLE | MUSCLE |
| | RELAXANT ACTIVITY | |
| QUERCETIN-3-O-BETA-D- | ANTIHEPATOTOXIC ACTIVITY; | ANTIHEPATOTOXIC ACTIVITY; |
| GLUCURONOPYRANOSIDE | GLUTAMATE-PYRUVATE- | HEPATOPROTECTION; |
| (FLAVONOID) | TRANSAMINASE INHIBITION; | ANTICANCER |
| | GLUTATHIONE-S-TRANSFERASE | |
| | INDUCTION | |
| QUERCETIN-3-O-BETA-D- | REVERSE TRANSCRIPTASE INHIBITION | ANTICANCER |
| XYLOPYRANOSIDE (FLAVONOID) | | |
| QUERCETIN-3-O-BETA-D- | CHROMOSOME ABERRATION INHIBITION | ANTICANCER |
| SYLOPYRANOSYL)(1-2)-BETA-D- | | |
| GLUCOPYRANOSIDE (FLAVONOID) | | |
| QUERCETIN-D-O-BETA- | CYTOTOXIC ACTIVITY | ANTICANCER |
| GLUCLIRONIDE (FLAVONOID) | | |
| QUERCETIN-3-O-D-ROBINOSIDE | ANTIOXIDANT ACTIVITY | ANTIOXIDANT |
| (FLAVONOID) | | |
| QUERCETIN-3-O-GALACTOSIDE | PLATELET AGGREGATION INHIBITION | PLATELET AGGREGATION |
| OCTAACETATE | | |
| QUERCETIN-3-O-GALACTOSYL-(1- | ANTIBACTERIAL ACTIVITY | ANTIBACTERIAL ACTIVITY |
| 6)-GLUCOSIDE (FLAVONOID) | | |
| QUERCETIN-3-O-GENTIOBIOSIDE | SPASMOLYTIC ACTIVITY | NERVOUS SYSTEM |
| (FLAVONOID) | | |
| QUERCETIN-3-O-GLUCO-RHAMNO- | ANTIINFLAMMATORY ACTIVITY | ANTIINFLAMMATORY ACTIVITY |
| ARABINOSIDE (FLAVONOID) | | |
| QUERCETIN-X-O-GLUCOSIDE | ANTIBACTERIAL ACTIVITY; XANTHINE | ANTIBACTERIAL ACTIVITY; |
| | OXIDASE INHIBITION | ANTIOXIDANT |
| QUERCETIN-3-O-GLUCOSIDE-2″-O- | CYTOTOXIC ACTIVITY; HISTAMINE | ANTICANCER; ANTIOXIDANT |
| GALLATE (FLAVONOID) | RELEASE INHIBITION | |
| QUERCETIN-3-O-GLUCURONIDE | CYTOTOXIC ACTIVITY; HISTAMINE | ANTICANCER; ANTIOXIDANT |
| (FLAVONOID) | RELEASE INHIBITION | |
| QUERCETIN-3-O-METHYL-4-O- | CYTOTOXIC ACTIVITY; HISTAMINE | ANTICANCER; ANTIOXIDANT |
| GLUCOSIDE | RELEASE INHIBITION | |
| QUERCETIN-3-O- | CELL PROLIFERATION INHIBITION; | ANTICANCER; PLATELET |
| NEOHESPERIDOSIDE (FLAVONOID) | PLASMINOGEN ACTIVATION | AGGREGATION; |
| | STIMULATION; PROSTAGLANDIN | ANTIINFLAMMATORY |
| | INDUCTION | |
| QUERCETIN-3-O- | ANTIINFLAMMATORY ACTIVITY; | ANTIINFLAMMATORY |
| RHAMNOGLUCOSIDE (FLAVONOID) | MOLLUSCICIDAL ACTIVITY | ACTIVITY; MOLLUSCICIDAL |
| | | ACTIVITY |
| QUERCETIN-3-O-RHAMNOSYL-(1-6)- | ANTIBACTERIAL ACTIVITY | ANTIBACTERIAL ACTIVITY |
| GLUCOSIDE (FLAVONOID) | | |
| QUERCETIN-3-O-RUTINOSIDE | ANTIPRURITIC ACTIVITY; COMPLEMENT | ALLERGY; IMMUNITY |
| (FLAVONOID) | ALTERNATIVE PATHWAY INHIBITION; | |
| | COMPLEMENT CLASSICAL PATHWAY | |
| | INHIBITION | |
| QUERCETIN-3-O-SOPHOROSIDE | LIPID PEROXIDE FORMATION INHIBITION | ANTIOXIDANT |
| QUERCETIN-3-O-XYLOSYL(1-2)- | ANTIINFLAMMATORY ACTMTY | ANTIINFLAMMATORY ACTIVITY |
| RHAMNOSIDE | | |
| QUERCETIN-3-RHAMNOSIDE-7- | ANTIOXIDANT ACTIVITY; | ANTIOXIDANT; |
| GLUCOSIDE | CYCLOOXYGENASE STIMULATION; | ANTIINFLAMMATORY; |
| | LIPOXYGENASE INHIBITION | ANTIOXIDANT |
| QUERCETIN-3-SULFATE | ANTIOXIDANT ACTIVITY; SUPEROXIDE | ANTIOXIDANT |
| | RADICAL SCAVENGING ACTIVITY | |
| QUERCETIN-4′-7-DIMETHYL ETHER | ANTIVIRAL ACTIVITY(PLANT | ANTIVIRAL ACTIVITY; |
| | PATHOGENS); COMPLEMENT | IMMUNITY |
| | ALTERNATIVE PATHWAY INHIBITION; | |
| | COMPLEMENT CLASSICAL PATHWAY, | |
| | INHIBITION | |
| QUERCETIN-4′-GLUCOSIDE | ANTIOXIDANT ACTIVITY; LIPID | ANTIOXIDANT; ANTICANCER |
| | PEROXIDE FORMATION INHIBITION; | |
| | QUINONE REDUCTASE INDUCTION | |
| QUERCETIN-4-O-BETA- | ANTIOXIDANT ACTIVITY | ANTIOXIDANT |
| GLUCOPYRANOSIDE-6″-GALLATE | | |
| QUERCETIN-4′-O- | ANTIOXIDANT ACTIVITY | ANTIOXIDANT |
| GLUCOPYRANOSYL-6″-GALLATE | | |
| QUERCETIN-6-METHOXY: 3-3- | ANTIVIRAL ACTIVITY | ANTIVIRAL ACTIVITY |
| DIMETHYL ETHER | | |
| QUERCETIN-ACETYL-TRISULFATE | ALCOHOL DEHYDROGENASE | HYPERTENSION; ANTIOXIDANT |
| | INHIBITION; LACTATE | |
| | DEHYDROGENASE INHIBITION; MALATE | |
| | DEHYDROGENASE INHIBITION | |
| QUERCETIN-RHAMNOSIDE | ANTIULCER ACTIVITY; SMOOTH MUSCLE | ANTIULCER; MUSCLE |
| | RELAXANT ACTIVITY | |
| QUERCITRIN | ADENOSINE A-1 RECEPTOR BINDING | MUSCLE ACTIVITY; |
| | INHIBITION; ALCOHOL | HYPERTENSION; |
| | DEHYDROGENASE INHIBITION; ALDOL | ANTINEOPLASTIC |
| | REDUCTASE (LENS) INHIBITION | |
| TANIN | ANTIBACTERIAL ACTIVITY; ANTIFUNGAL | HORMONE; ANTIBACTERIAL |
| | ACTIVITY; ANTIHCG ACTIVITY; | ACTIVITY; ANTIFUNGAL |
| | ANTIHISTAMINE ACTIVITY; | ACTIVITY; HORMONE; |
| | ANTIINFLAMMATORY ACTIVITY; | ALLERGY; |
| | ANTIOXIDANT ACTIVITY; ANTI- | ANTIINFLAMMATORY |
| | PREGNANT MARE SERUM | ACTIVITY; ANTIOXIDANT; |
| | GONADOTROPIN ACTIVITY; ANTITUMOR | HORMONE; ANTITUMOR |
| | ACTIVITY; CALCIUM ION UPTAKE | ACTIVITY; BLOOD; |
| | STIMULATION; CYTOTOXIC ACTIVITY; | ANTICANCER; FERTILITY; |
| | ESTROUS CYCLE DISRUPTION EFFECT; | INSECTICIDAL; LIVER; |
| | FEEDING DETERRENT (INSECT); | ANTITUMOR; IMMUNITY; |
| | FEEDING SSTIMULANT (INSECT); | FERTILITY |
| | HEPATOTOXIC ACTIVITY; NO-SYNTHASE | |
| | INHIBITION; ORNITHINE | |
| | DECARBOXYLASE INHIBITION; | |
| | POLYGLYCOHYDROLASE INHIBITION; | |
| | UTERINE STIMULANT EFFECT | |
| URSOLIC ACID | ACID PHOSPHATASE INHIBITION; | ANTINEOPLASTIC; |
| | ADENOSINE DEAMINASE INHIBITION | IMMUNODEFICIENCY |

Equivalents

Those skilled in the art will recognize, or be able to ascertain using no more than routine experimentation, many equivalents to the specific embodiments of the invention described herein. Such equivalents are intended to be encompassed by the following claims.

What is claimed:

1. A method for treating or preventing a malignancy or hypercholesterolemia in a subject comprising,
administering to said subject a therapeutically-effective amount of a composition selected from the group consisting of an essence oil isolated from a citrus fruit, a peel oil isolated from a citrus fruit, a peel isolated from a citrus fruit, decharacterized cranberry fruit, and combinations thereof.

2. The method of claim 1, wherein the malignancy is breast cancer.

3. The method of claim 1, wherein the malignancy is metastatic.

4. The method of claim 1, wherein the hypercholesterolemia is measured by altered apoB levels.

5. The method of claim 1, wherein the composition further comprises a compound selected from group comprising fats, carbohydrates, proteins, vitamins, minerals, and combinations thereof.

6. The method of claim 1, wherein the composition is administered orally.

7. The method of claim 1, wherein the citrus fruit is a grapefruit.

8. A method of nutritionally supplementing a foodstuff comprising,
adding to said foodstuff a composition selected from the group consisting of an essence oil isolated from a citrus fruit, a peel oil isolated from a citrus fruit, a peel isolated from a citrus fruit, decharacterized cranberry fruit, and combinations thereof.

9. A dietary supplement comprising, a composition selected from the group consisting of an essence oil isolated from a citrus fruit, a peel oil isolated from a citrus fruit, a peel isolated from a citrus fruit, decharacterized cranberry fruit, and combinations thereof.

10. A method of supplementing a pharmaceutical comprising,
adding to said pharmaceutical a composition selected from the group consisting of an essence oil isolated from a citrus fruit, a peel oil isolated from a citrus fruit, a peel isolated from a citrus fruit, decharacterized cranberry fruit, and combinations thereof.

11. A composition comprising a compound isolated from cranberry and selected from the group consisting of a phenolic acid, flavanoid, fiber, omega-3-fatty acid, tocochromanol, triterpenoid, ellagic acid, and combinations thereof.

12. A composition comprising a compound isolated from cranberry and selected from the group consisting of an anthocyanin, a phenolic acid, a proanthocyanidin, and combinations thereof.

13. A composition isolated from a cranberry having an anthocyanin content which is 30% or greater of that present in the native fruit, a phenolic acid content of 8% or greater of that present in the native fruit, and a proanthocyanidin content of 60% or greater of that present in the native fruit.

14. The composition of claim 11, wherein said phenolic acid is selected from the group consisting of para-coumaric acid, caffeic acid, chlorogenic acid, ferulic acid, protocatechuic acid, cinnamic acid, benzoic acid, gallic acid, para-hydroxybenzoic acid, and combinations thereof.

15. The composition of claim 11, wherein said flavanoid is selected from the group consisting of a proanthocyanidin, flavan-3-ol, anthocyanin, flavanol, and combinations thereof.

16. The composition of claim 15, wherein said proanthocyanidin is selected from the group consisting of a flavan-3-ol polymer, procyanidin B 1, procyanidin B 2, procyanidin B 3, epicatechin oligomer, and combinations thereof.

17. The composition of claim 15, wherein said flavan-3-ol is selected from the group consisting of catechin, catechin gallate, epicatechin, epicatechin gallate, epigallocatechin gallate, gallocatechin gallate, and combinations thereof.

18. The composition of claim 15, wherein said anthocyanin is selected from the group consisting of cyanidin-3-arabinoside, cyanidin-3-galactoside, cyanidin-3-glucoside, peonidin-3-arabinoside, peonidin-3-galactoside, peonidin-3-glucoside, malvidin-3-arabinoside, malvidin-3-glucoside, and combinations thereof.

19. The composition of claim 15, wherein said flavanol is selected from the group consisting of quercetin, q-3-arabinoside (avicularin), q-3-galactoside (hyperin), q-3-glucoside (isoquercitrin), q-3-rhamnoside (quercitrin), myricetin, m-3-arabinoside, m-3-rhamnoside (myricitrin), m-3-digalactoside, kaempferol, isorhamnetin, and combinations thereof.

20. The composition of claim 11, wherein said triterpenoid is ursolic acid.

21. A foodstuff comprising a composition according to claim 11.

22. A dietary supplement comprising a composition according to claim 11.

23. A pharmaceutical comprising a composition according to claim 11.
